SEQUENCE LISTING (8 sequences)

SEQ ID NO: 1 - DNA, 22 bp, synthetic construct
  source 1..22: mol_type = other DNA
caagatggattgcacgcaggtt

SEQ ID NO: 2 - DNA, 22 bp, synthetic construct
  source 1..22: mol_type = other DNA
tagaaggcgatgcgctgcgaat

SEQ ID NO: 3 - DNA, 21 bp, synthetic construct
  source 1..21: mol_type = other DNA
taaacggccacaagttcagcg

SEQ ID NO: 4 - DNA, 21 bp, synthetic construct
  source 1..21: mol_type = other DNA
aggaccatgtgatcgcgcttc

SEQ ID NO: 5 - DNA, 20 bp, synthetic construct
  source 1..20: mol_type = other DNA
atgaaggttatcgctgtact

SEQ ID NO: 6 - DNA, 20 bp, synthetic construct
  source 1..20: mol_type = other DNA
ctgattcaagaacggttgtg

SEQ ID NO: 7 - DNA, 21 bp, synthetic construct
  source 1..21: mol_type = other DNA
ctcctgctaaggtatataagc

SEQ ID NO: 8 - DNA, 20 bp, synthetic construct
  source 1..20: mol_type = other DNA
aatcaggcttgatccccagt

(19) United States
(12) Patent Application Publication
(10) Pub. No.: US 2025/0122514 A1
(43) Pub. Date: Apr. 17, 2025

(54) VECTORS AND METHODS FOR IMPROVED DICOT PLANT TRANSFORMATION FREQUENCY

(71) Applicant: THE REGENTS OF THE UNIVERSITY OF CALIFORNIA, Oakland, CA (US)

(72) Inventors: Myeong-Je Cho, Oakland, CA (US); Brian Staskawicz, Oakland, CA (US)

(73) Assignee: THE REGENTS OF THE UNIVERSITY OF CALIFORNIA, Oakland, CA (US)

(21) Appl. No.: 18/704,408

(22) PCT Filed: Oct. 25, 2022
(86) PCT No.: PCT/US2022/047731

§ 371 Date: Apr. 24, 2024

Related U.S. Application Data
(60) Provisional application No. 63/271,654, filed on Oct. 25, 2021.

(51) Int. Cl. C12N 15/82

(52) U.S. Cl. CPC C12N 15/8222; C12N 15/8205

(57) ABSTRACT

In certain embodiments, provided are methods for increasing the efficiency of dicot and gymnosperm transformation using BBM with no or minimal plant phenotype. Vector designs for achieving these results are also provided, as are methods for their use.

### CROSS-REFERENCE TO RELATED APPLICATION

This application is a 35 U.S.C. § 371 application of International Application Serial No. PCT/US2022/047731, filed Oct. 25, 2022, and claims priority to U.S. Provisional Application No. 63/271,654, filed Oct. 25, 2021, the contents of which are incorporated by reference herein.

### SEQUENCE LISTING

The instant application contains a Sequence Listing which has been submitted electronically in XML file format and is hereby incorporated by reference in its entirety. Said XML copy, created on Nov. 13, 2024, is named 19520003US1.xml and is 8,205 bytes in size.

### BACKGROUND

Morphogenic genes, BabyBoom (BBM) and Wuschel (WSU) together or individually, have been used for increasing the efficiency of monocot transformation. The use of these genes for dicot transformation was difficult because of an abnormal phenotype and regeneration issue, especially BBM was not successfully demonstrated. Accordingly, methods are needed for increasing the efficiency of dicot transformation using BBM with no or minimal plant phenotype. Methods are need also for increasing the efficiency of gymnosperm transformation using BBM with no or minimal plant phenotype.

### SUMMARY

Certain embodiments of the invention provide a transformed plant cell comprising a heterologous nucleic acid construct comprising a promoter and a transcription factor related to embryogenesis and/or organogenesis (such as a morphogenic gene transcription factor or developmental gene transcription factor), wherein the promoter is a plant embryo-specific or seed-specific promoter.

Certain embodiments of the invention provide a nucleic acid construct comprising a plant embryo-specific promoter and a transcription factor related to embryogenesis and/or organogenesis, wherein the transcription factor is BabyBoom (BBM) or Wuschel (WSU).

Certain embodiments of the invention provide a nucleic acid construct comprising pGmLe1AtWUS or pGmLe1AtBBM.

Certain embodiments of the invention provide a method of transforming plant tissue, comprising contacting the plant tissues with a heterologous nucleic acid construct comprising a promoter and a transcription factor related to embryogenesis and/or organogenesis (such as a morphogenic gene transcription factor or developmental gene transcription factor), wherein the promoter is a plant embryo-specific or seed-specific promoter.

Certain embodiments of the invention provide a method of increasing seed production in a plant compared to a non-transformed plant, comprising contacting the plant tissues with a heterologous nucleic acid construct comprising a promoter and a transcription related to embryogenesis and organogenesis factor (such as a morphogenic gene transcription factor or developmental gene transcription factor), wherein the promoter is a plant embryo-specific or seed-specific promoter.

### BRIEF DESCRIPTION OF DRAWINGS

FIGS. 1A-1B. Schematic diagram of two transformation vectors used for Theobroma cacao L. transformation. (a) pDDNPTYFP-1 is a 3637-bp T-DNA fragment containing neomycin phosphotransferase II (nptII) and enhanced yellow fluorescent protein (eyfp) translational fusion driven by a CaMV35S promoter. (b) pDDNPTYFP-2 is a 4416-bp T-DNA fragment containing two gene cassettes where eyfp is driven by a Nos promoter/TMV Ω enhancer and nptII is driven by an enhanced CaMV35S promoter.

FIG. 2. Somatic embryo formation from flower materials for 9 different Theobroma cacao L. cultivars. Petal and staminode explants were used to induce primary somatic embryos. Each histogram represents the mean level (±standard error).

FIGS. 3A-3B. Secondary somatic embryogenesis and embryo conversion to plantlets for selected Theobroma cacao L. cultivars. (a) Cotyledon explants were observed in four ranks based on the number of embryos they produced: No embryos produced, 1 to 4 embryos produced, 5 to 9 embryos produced, and greater than 10 embryos produced. (b) Mature embryos were exposed to increasing levels of light while on embryo development medium containing 6% sucrose then embryo development light medium and counted as converted if they produced a 1 cm shoot with meristem and a root of 1 cm. Each histogram represents the mean level (±standard error).

FIG. 4. Effect of doubled woody plant medium (WPM) basal salt concentration and WPM adjusted to 5.0μ of CuSO₄ in secondary callus generation (SCG) medium on secondary somatic embryogenesis in Theobroma cacao L. cv. INIAPG-038. Cotyledon explants were observed in four ranks based on the number of embryos they produced: No embryos produced, 1 to 4 embryos produced, 5 to 9 embryos produced, and greater than 10 embryos produced. Each histogram represents the mean level (±standard error).

FIG. 5. Transient yellow fluorescent protein (YFP) expression for selected Theobroma cacao L. cultivars. Cotyledon explants were observed in three ranks based on fluorescent protein expression (FTE) coverage: 0 to 5% FPE coverage, 6 to 20% FPE coverage, >20% FPE coverage. Each histogram represents the mean level (±standard error).

FIG. 6. Effect of Agrobacterium strain on transient yellow fluorescent protein (YFP) expression in Theobroma cacao L. cv. INIAPG-038. Cotyledon explants were observed in three ranks based on fluorescent protein expression (FTE) coverage: 0 to 5% FPE coverage, 6 to 20% FPE coverage, >20% FPE coverage. Each histogram represents the mean level (±standard error).

FIG. 7. Effect of somatic embryo size on transient yellow fluorescent protein (YFP) expression in Theobroma cacao L. cv. INIAPG-038. Cotyledon tissue derived from somatic embryos was used for experiments. Cotyledon explants were observed in three ranks based on fluorescent protein expression (FTP) coverage: 0 to 5% FPE coverage, 6 to 20% FPE coverage, >20% FPE coverage. Each histogram represents the mean level (±standard error).

FIGS. 8A-8G. Transient yellow fluorescent protein (YFP) expression and stable transgenic event development in Theobroma cacao L. cv. INIAPG-038. (a) Cotyledon expressing transient YFP. (b) YFP-expressing globular embryo. (c) YFP-expressing mature embryo. (d) YFP-expressing quaternary somatic embryos. (e) YFP-expressing plantlet. (f) A non-transgenic (leftmost) and 3 transgenic INIAPG-038 plants in the greenhouse. (g) A grafted transgenic INIAPG-038 plantlet in a Jiffy peat pellet; a transgenic INIAPG-038 scion with poor roots was grafted onto a non-transgenic rootstock.

FIGS. 9A-9D. PCR analysis of genomic DNA from three different transgenic Theobroma cacao L. cv. INIAPG-038 events. Genomic DNA was extracted from leaf tissues of one non-transgenic control and three plants of each independent transgenic event. M GeneRuler 1-kb DNA ladder, C Non-transgenic INIAPG-038 control, Lanes 1-3 Transgenic INIAPG-038 Event #1-1, #1-2 and #1-3, Lanes 4-6 Transgenic INIAPG-038 Event #2-1, #2-2 and #2-3, Lanes 7-9 Transgenic INIAPG-038 Event #3-1, #3-2 and #3-3, P pDDNPTYFP-2 as a positive control. (a) Amplified 595-bp fragment products using enhanced yellow fluorescent protein (eyfp) gene-specific primers, EYFP 3F and EYFP 4R. (b) Amplified 761-bp fragment products using neomycin phosphotransferase II (nptII) gene-specific primers, NPTII 3F and NPTII 4R (c) Amplified 562-bp fragment products using pVS1 replicon-specific primers, PVS1 1F and PVS1 2R. (d) Amplified 672-bp fragment products using neomycin phosphotransferase I (npt1) gene-specific primers, NPT1 1F and NPT1 2R.

FIGS. 10A-10E. Schematic diagram of five transformation vectors used for cacao transformation. (A) control vector, (B) pGmLe1AtBBM, (C) pGmLe1AtWUS, (D) pGmLe1VvGRF/GIF, and (E) pZmUbi1VvGRF/GIF.

FIG. 11. Generation of transgenic cacao somatic embryos using pGmLe1AtBBM.

FIG. 12. Putative transgenic tobacco shoots 30 days after Agrobacterium infection.

FIG. 13. Putative transgenic Nicotiana benthamiana shoots 30 days after Agrobacterium infection.

FIG. 14. Putative transgenic tobacco shoots 66 days after Agrobacterium infection.

FIG. 15. Putative transgenic Nicotiana benthamiana shoots 66 days after Agrobacterium infection.

FIG. 16. Transgenic tobacco plants transformed with pGmLe1AtBBM and pGmLe1AtWUS show normal phenotype. Photos were taken 66 days after Agrobacterium infection.

FIGS. 17A-17B. Yellow fluorescent protein (YFP) expression in (a) leaf disks of transgenic Theobroma cacao L. cv. INIAPG-038 events and (b) Agrobacterium strains containing vectors including the enhanced yellow fluorescent protein gene cassette used for INIAPG-038 transformation. Top, Brightfield; Bottom, YFP filter. (a) Leaf disks in order from left-most column to right-most: non-transgenic INIAPG-038, Event #1, Event #2, Event #3. Each row was sampled from different leaf or plant. (b) Agrobacterium strains in order from left to right: AGL1 without a T-DNA binary vector, AGL1 with pDDNPTYFP-1, AGL1 with pDDNPTYFP-2.

FIG. 18. Constructs pGmLe1pAtBBM and pGmLe1pAtWUS.

FIGS. 19A-19C. Germinating cacao somatic embryos transformed with GmLel1AtBBM having a normal phenotype. FIG. 19A Matina 1-6. FIG. 19B INIAPG-038. FIG. 19C. CCN-51.

FIG. 20. Matina 1-6 Plantlets transformed with pGmLe1AtBBM having a normal plant phenotype.

FIG. 21. Non-regenerable cacao callus formation transformed with pGmLe1AtWUS.

FIG. 22. Formation of multiple somatic embryos from a single INIAPG-038 embryo transformed with pGmLe1AtBBM.

FIG. 23. Matina 1-6 somatic embryos transformed with pGmLe1AtBBM having an abnormal phenotype.

FIG. 24. Transgenic tobacco plants transformed with control, pGmLe1AtBBM and pGmLe1AtWUS having a normal phenotype.

FIG. 25. Formation of non-regenerable M82 green callus tissue transformed with pGmLe1AtWUS.

FIG. 26. Transgenic M82 tomato plants transformed with control, pGmLe1AtBBM and pGmLe1AtWUS having a normal phenotype. The control plant was transferred to soil at a later date than the other plants.

FIGS. 27A-27D. Transgenic cassava plants transformed with control, pGmLe1AtBBM, pGmLe1AtWUS and pZmUbi1 VvGRF/GIF having a normal phenotype. FIG. 27A. Control. FIG. 27B. pGmLe1AtBBM. FIG. 27C. pGmLe1AtWUS. FIG. 27D. pZmUbiVvGRF/GIF.

### DETAILED DESCRIPTION

The present disclosure related to the field of plant transformation. Provided are methods for increasing the efficiency of dicot transformation using transcription factor related to embryogenesis and/or organogenesis genes with no or minimal plant phenotype. Vector designs for achieving these results are also provided, as are methods for their use.

A highly efficient transformation protocol is a prerequisite to developing genetically modified and genome-edited crops. The present invention is useful as a transformation and genome editing tool especially for recalcitrant dicot crops and cultivars.

Certain embodiments of the invention provide a transformed plant cell comprising a heterologous nucleic acid construct comprising a promoter and a transcription factor related to embryogenesis and/or organogenesis, wherein the promoter is a plant embryo-specific or seed-specific promoter.

In certain embodiments, the plant cell is a dicotyledon cell.

In certain embodiments, the dicotyledon cell is a cacao, cassava, soybean, tomato, tobacco, coffee, sunflower, alfalfa, pepper, cowpea, sunflower, grape, poplar, coffee, sunflower, walnut, almond, peach, nectarine, plum, apricot, apple, pear, persimmon, or other fruit tree cell.

In certain embodiments, the plant cell is a gymnosperm cell.

In certain embodiments, the gymnosperm cell is selected from pine, ginkgo, spruce, or Douglas fir cells.

In certain embodiments, the promoter is an inducible promoter.

In certain embodiments, the promoter is a plant embryo-specific promoter, and the plant embryo-specific promote is a dicot embryo-specific promoter.

In certain embodiments, the dicot embryo-specific promoter is a soybean embryo-specific promoter.

In certain embodiments, the soybean embryo-specific promoter is a soybean lectin promoter.

In certain embodiments, the soybean embryo-specific promoter is a soybean glycinin promoter.

In certain embodiments, the soybean embryo-specific promoter is a soybean conglycinin promoter.

In certain embodiments, the promoter is a plant seed-specific promoter, and the plant seed-specific promote is a dicot seed-specific promoter.

In certain embodiments, the dicot seed-specific promoter is a soybean seed-specific promoter.

In certain embodiments, the transcription factor related to embryogenesis and/or organogenesis is a morphogenic gene transcription factor or developmental gene transcription factor.

In certain embodiments, the transcription factor is BabyBoom (BBM).

In certain embodiments, the construct further comprises a vector.

In certain embodiments, the vector is a plasmid.

In certain embodiments, the construct further comprises a site-specific recombinase.

In certain embodiments, the construct further comprises a trait gene. As used herein, a “trait gene” can be a gene product conferring nutritional enhancement, pharmaceutical protein production, increased seed or biomass yield, abiotic stress tolerance, drought tolerance, cold tolerance, herbicide tolerance, pest resistance, insect resistance, disease resistance, nitrogen use efficiency (NUE), or an ability to alter a metabolic pathway.

In certain embodiments, the construct further comprises a selectable/visual marker gene.

In certain embodiments, the construct further comprises a CRISPR cassette.

Certain embodiments of the invention provide a nucleic acid construct comprising a plant embryo-specific promoter and a transcription factor, wherein the transcription factor is BabyBoom (BBM) or Wuschel (WSU).

In certain embodiments, the transcription factor is BabyBoom (BBM).

In certain embodiments, the transcription factor is Wuschel (WSU).

Certain embodiments of the invention provide a nucleic acid construct comprising pGmLe1AtWUS or pGmLe1AtBBM.

Certain embodiments of the invention provide a method of transforming plant tissue, comprising contacting the plant tissue with a heterologous nucleic acid construct comprising a promoter and a transcription factor, wherein the promoter is a plant embryo-specific or seed-specific promoter.

In certain embodiments, the plant tissue is dicotyledon tissue.

In certain embodiments, the dicotyledon tissue is a cacao, cassava, soybean, tomato, tobacco, coffee, sunflower, alfalfa, pepper, cowpea, sunflower, grape, poplar, coffee, sunflower, walnut, almond, peach, nectarine, plum, apricot, apple, pear, persimmon, or other fruit tree cell.

In certain embodiments, the plant tissue is gymnosperm tissue.

In certain embodiments, the gymnosperm tissue is selected from pine, ginkgo, spruce, or Douglas fir tissue.

In certain embodiments, the plant tissue is zygotic embryo tissue.

In certain embodiments, the plant tissue is somatic embryo tissue.

In certain embodiments, the plant tissue is somatic embryogenic callus tissue.

In certain embodiments, the plant tissue is organogenic tissue.

In certain embodiments, the plant tissue is shoot meristematic tissue.

In certain embodiments, the plant tissue is geminating seedling tissue.

In certain embodiments, the plant tissue is leaf tissue.

In certain embodiments, the plant tissue is protoplast-derived tissue.

### Aspects of the Invention

One aspect of the invention is a transformed dicotyledon or gymnosperm plant cell comprising a heterologous nucleic acid construct comprising a plant seed-specific promoter and a morphogenic gene transcription factor BabyBoom (BBM).

In one aspect, the plant cell is a dicotyledon.

In one aspect, the dicotyledon cell is a cacao, cassava, soybean, tomato, tobacco, alfalfa, pepper, cowpea, sunflower, grape, poplar, coffee, walnut, almond, peach, nectarine, plum, apricot, apple, pear, persimmon, or other fruit tree cell.

In one aspect, the plant cell is a gymnosperm cell.

In one aspect, the gymnosperm is selected from a pine, ginkgo, spruce, or Douglas fir cell.

In one aspect, the promoter is a plant seed-specific promoter.

In one aspect, the plant seed-specific promoter is a cotyledon seed-specific promoter.

In one aspect, the cotyledon seed-specific promoter is a seed storage protein gene promoter.

In one aspect, the seed storage protein gene promoter a conglycinin, glycinin, lectin, phaseolin, legumin, vicilin, convicilin, napin or cruciferin protein gene promoter.

In one aspect, the cotyledon seed-specific promoter is an embryo-specific promoter.

In one aspect, the embryo-specific promoter is a soybean seed-specific promoter.

In one aspect, the soybean seed-specific promoter is a soybean cotyledon-specific promoter.

In one aspect, the soybean cotyledon-specific promoter is a soybean seed storage protein gene promoter.

In one aspect, the soybean cotyledon-specific promoter is a soybean embryo-specific promoter.

In one aspect, the soybean embryo-specific promoter is a soybean lectin promoter.

In one aspect, the soybean embryo-specific promoter is a soybean glycinin promoter.

In one aspect, the soybean embryo-specific promoter is a soybean conglycinin promoter.

In one aspect, the construct further comprises a vector.

In one aspect, the vector is a plasmid.

In one aspect, the construct further comprises a site-specific recombinase.

In one aspect, the construct further comprises a trait gene.

In one aspect, the construct further comprises a selectable/visual marker gene.

In one aspect, the construct further comprises a CRISPR cassette.

One aspect of the invention is a nucleic acid construct comprising a plant seed-specific promoter and a morphogenic gene transcription factor BabyBoom (BBM).

One aspect of the invention is a nucleic acid construct comprising pGmLe1AtBBM.

One aspect of the invention is a method of transforming dicotyledon or gymnosperm plant tissue, comprising contacting the plant tissue with a heterologous nucleic acid construct comprising a plant seed-specific promoter and a morphogenic gene transcription factor BabyBoom (BBM).

In one aspect, the plant tissue is dicotyledon tissue.

In one aspect, the dicotyledon tissue is a cacao, cassava, soybean, tomato, tobacco, alfalfa, pepper, cowpea, sunflower, grape, poplar, coffee, walnut, almond, peach, nectarine, plum, apricot, apple, pear, persimmon, other fruit tree tissue.

In one aspect, the tissue is gymnosperm tissue.

In one aspect, the gymnosperm tissue is selected from pine, ginkgo, spruce, or Douglas fir tissue.

In one aspect, the plant tissue is zygotic embryo.

In one aspect, the plant tissue is somatic embryo.

In one aspect, the plant tissue is somatic embryogenic callus tissue.

In one aspect, the plant tissue is organogenic tissue.

In one aspect, the plant tissue is shoot meristematic tissue.

In one aspect, the plant tissue is geminating seedling.

In one aspect, the plant tissue is leaf tissue.

In one aspect, the plant tissue is protoplast-derived tissue.

### Nucleic Acids, Expression Cassettes, Vectors and Cells

Certain embodiments of the invention provide a nucleic acid encoding a promoter and a transcription factor related to embryogenesis and/or organogenesis as described herein.

Certain embodiments of the invention provide an expression cassette comprising a nucleic acid as described herein and a promoter.

Certain embodiments of the invention provide a vector (e.g., a plasmid) comprising an expression cassette as described herein.

Certain embodiments of the invention provide a cell comprising a vector as described herein.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res., 19:508 (1991); Ohtsuka et al., JBC, 260:2605 (1985); Rossolini et al., Mol. Cell. Probes, 8:91 (1994). A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

By “portion” or “fragment,” as it relates to a nucleic acid molecule, sequence or segment of the invention, when it is linked to other sequences for expression, is meant a sequence having at least 80 nucleotides, more specifically at least 150 nucleotides, and still more specifically at least 400 nucleotides. If not employed for expressing, a “portion” or “fragment” means at least 9, specifically 12, more specifically 15, even more specifically at least 20, consecutive nucleotides, e.g., probes and primers (oligonucleotides), corresponding to the nucleotide sequence of the nucleic acid molecules of the invention.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, culture medium may represent less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance, the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, NY: Cold Spring Harbor Laboratory Press (3^{rd} edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Wild-type” refers to the normal gene, or organism found in nature without any known mutation.

“Genome” refers to the complete genetic material of an organism.

A “vector” is defined to include, inter alia, any plasmid, cosmid, phage or binary vector in double or single stranded linear or circular form which may or may not be self-transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

Generally, the expression cassette will comprise a selectable marker gene for the selection of transformed cells. Selectable marker genes are utilized for the selection of transformed cells or tissues. Marker genes include genes encoding antibiotic resistance. Such as those encoding neomycin phosphotransferase II (nptII) and hygromycin phosphotransferase (HPT), as well as genes conferring resistance to herbicidal compounds, such as glufosinate ammonium, bromoxynil, imidazolinones, glyphosate and 2,4-dichlorophenoxyacetate (2,4-D). Any selectable marker gene can be used in the present invention.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., Mol. Biotech., 3:225 (1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule.

The terms “trans-acting sequence” and “trans-acting element” refer to DNA or RNA sequences whose function does not require them to be on the same molecule.

The following terms are used to describe the sequence relationships between two or more sequences (e.g., nucleic acids, polynucleotides or polypeptides): (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA, gene sequence or peptide sequence, or the complete cDNA, gene sequence or peptide sequence.

(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, JMB, 48:443 (1970); the search-for-similarity-method of Pearson and Lipman, Proc. Natl. Acad. Sci. USA, 85:2444 (1988); the algorithm of Karlin and Altschul, Proc. Natl. Acad. Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to, CLUSTAL in the PC/Gene program (available from Intelligenetics, Mountain View, California); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wisconsin, USA). Alignments using these programs can be performed using the default parameters. The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more specifically less than about 0.01, and most specifically less than about 0.001.

To obtain gapped alignments for comparison purposes, Gapped BLAST (in BLAST 2.0) can be utilized as described in Altschul et al., Nucleic Acids Res. 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent and are different under different environmental parameters. Longer sequences hybridize specifically at higher temperatures. The thermal melting point (Tₘ) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267 (1984); Tₘ 81.5° C.+16.6(log M)+0.41(% GC)−0.61(% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology Hybridization with Nucleic Acid Probes, part I chapter 2 “Overview of principles of hybridization and the strategy of nucleic acid probe assays” Elsevier, New York (1993). Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more specifically about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism into which a heterologous nucleic acid molecule has been introduced. The nucleic acid molecule can be stably integrated into the genome generally known in the art and are disclosed in Sambrook and Russell, supra. See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

### Nucleic Acids

The present invention provides nucleic acids that encode portions or all of a protein. The term “nucleic acid” refers to deoxyribonucleic acid (DNA) or ribonucleic acid (RNA) and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a portion of a given nucleic acid molecule.

The terms “polynucleotide”, “nucleic acid” and “nucleic acid fragment” are used interchangeably herein. These terms encompass nucleotides connected by phosphodiester linkages. A “polynucleotide” may be a ribonucleic acid (RNA) or deoxyribonucleic acid (DNA) polymer that is single- or double-stranded, that optionally contains synthetic, non-natural or altered nucleotide bases. A polynucleotide in the form of a polymer of DNA may comprise one or more segments of cDNA, genomic DNA, synthetic DNA, or mixtures thereof. Nucleotide bases are indicated herein by a single letter code: adenine (A), guanine (G), thymine (T), cytosine (C), inosine (I) and uracil (U). Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

A “nucleotide sequence” is a polymer of DNA or RNA that can be single-stranded or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” are used interchangeably and may also be used interchangeably with gene, cDNA, DNA and RNA encoded by a gene.

The invention encompasses isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. Fragments and variants of the disclosed nucleotide sequences are also encompassed by the present invention. By “fragment” or “portion” is meant a full length or less than full length of the nucleotide sequence.

“Naturally occurring,” “native,” or “wild-type” is used to describe an object that can be found in nature as distinct from being artificially produced. For example, a protein or nucleotide sequence present in an organism (including a virus), which can be isolated from a source in nature and that has not been intentionally modified by a person in the laboratory, is naturally occurring.

“Genome” refers to the complete genetic material of an organism.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al. 1984), BLASTP, BLASTN, and FASTA (Altschul, S. F., et al., 1990. The BLASTX program is publicly available from NCBI and other sources (BLAST Manual, Altschul, S., et al., NCBI NLM NIH Bethesda, Md. 20894; Altschul, S., et al., 1990). The well-known Smith Waterman algorithm may also be used to determine identity.

Nucleic acid molecules encoding amino acid sequence variants of a protein are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the protein.

### Promoters

“Promoter” refers to a nucleotide sequence, usually upstream (5′) to its coding sequence, that controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. A general discussion of promoters is provided in U.S. Pat. No. 7,501,129, which is incorporated by reference herein.

The invention encompasses isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences are also encompassed by the present invention. By “fragment” or “portion” is meant a full length or less than full length of the nucleotide sequence.

“Naturally occurring,” “native,” or “wild-type” is used to describe an object that can be found in nature as distinct from being artificially produced. For example, a protein or nucleotide sequence present in an organism (including a virus), which can be isolated from a source in nature and that has not been intentionally modified by a person in the laboratory, is naturally occurring.

In certain embodiments, the present invention provides vectors and expression cassettes containing the promoters described above. A “vector” is defined to include, inter alia, any viral vector, as well as any plasmid, cosmid, phage or binary vector in double or single stranded linear or circular form that may or may not be self-transmissible or mobilizable, and that can transform prokaryotic or eukaryotic host either by integration into the cellular genome or exist extrachromosomally (e.g., autonomous replicating plasmid with an origin of replication).

Nucleic acids encoding therapeutic compositions can be engineered into a vector using standard ligation techniques, such as those described in Sambrook and Russell, Molecular Cloning: A Laboratory Manual, Cold Spring Harbor Laboratory Press Cold Spring Harbor, NY (2001). For example, ligations can be accomplished in 20 mM Tris-Cl pH 7.5, 10 mM MgCl2, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration).

In certain embodiments, the present invention provides a vector containing an expression cassette comprising a promoter operably linked to a target sequence (e.g., a protein of interest) for production of vaccine. “Expression cassette” as used herein means a nucleic acid sequence capable of directing expression of a particular nucleotide sequence in an appropriate host cell, which includes a promoter operably linked to the nucleotide sequence of interest that may be operably linked to termination signals. The coding region usually codes for a functional RNA of interest, for example an RNAi molecule. The expression cassette including the nucleotide sequence of interest may be chimeric.

“Operably-linked” refers to the association of nucleic acid or amino acid sequences on single molecular fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. Nucleic acid is “operably linked” when it is placed into a functional relationship with another nucleic acid sequence. Generally, “operably linked” means that the DNA sequences being linked are contiguous. However, enhancers do not have to be contiguous. Linking is accomplished by ligation at convenient restriction sites. If such sites do not exist, the synthetic oligonucleotide adaptors or linkers are used in accordance with conventional practice. Additionally, multiple copies of the nucleic acid encoding enzymes may be linked together in the expression vector. Such multiple nucleic acids may be separated by linkers.

“Expression” refers to the transcription and/or translation of an endogenous gene or a transgene in cells. For example, in the case of antisense constructs, expression may refer to the transcription of the antisense DNA only. In addition, expression refers to the transcription and stable accumulation of sense (mRNA) or functional RNA. Expression may also refer to the production of protein.

“Expression cassette” as used herein means a DNA sequence capable of directing expression of a particular nucleotide sequence in an appropriate host cell, comprising a promoter operably linked to the nucleotide sequence of interest that is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Such expression cassettes will comprise the transcriptional initiation region linked to a nucleotide sequence of interest. Such an expression cassette may be provided with a plurality of restriction sites for insertion of the gene of interest to be under the transcriptional regulation of the regulatory regions. The expression cassette may additionally contain selectable marker genes.

The present disclosure also provides a mammalian cell containing a vector described herein.

### Proteins

The terms “protein,” “peptide” and “polypeptide” are used interchangeably herein.

A protein if interest can be conjugated or linked to another peptide or to a polysaccharide.

The term “amino acid” includes the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in Dextrorotary or Levorotary stereoisomeric forms, as well as unnatural amino acids (e.g., phosphoserine, phosphothreonine, phosphotyrosine, hydroxyproline, and gamma-carboxyglutamate; hippuric acid, octahydroindole-2-carboxylic acid, statine, 1,2,3,4-tetrahydroisoquinoline-3-carboxylic acid, penicillamine, ornithine, citruline, alpha-methyl-alanine, para-benzoylphenylalanine, phenylglycine, propargylglycine, sarcosine, and tert-butylglycine). The term also comprises natural and unnatural amino acids (Dextrorotary and Levorotary stereoisomers) bearing a conventional amino protecting group (e.g. acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc, and documents cited therein). An amino acid can be linked to the remainder of a compound through the carboxy terminus, the amino terminus, or through any other convenient point of attachment, such as, for example, through the sulfur of cysteine.

The invention encompasses isolated or substantially purified protein compositions. In the context of the present invention, an “isolated” or “purified” polypeptide is a polypeptide that exists apart from its native environment and is therefore not a product of nature. A polypeptide may exist in a purified form or may exist in a non-native environment such as, for example, a transgenic host cell. For example, an “isolated” or “purified” protein, or biologically active portion thereof, is substantially free of other cellular material, or culture medium when produced by recombinant techniques, or substantially free of chemical precursors or other chemicals when chemically synthesized. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed proteins or partial-length proteins encoded thereby are also encompassed by the present invention. By “fragment” or “portion” is meant a full length or less than full length of the amino acid sequence of, a polypeptide or protein.

A “variant” of a molecule is a sequence that is substantially similar to the sequence of the native molecule.

“Isolated” means altered or removed from the natural state. For example, a nucleic acid or a peptide naturally present in a living animal is not “isolated.” but the same nucleic acid or peptide partially or completely separated from the coexisting materials of its natural state is “isolated.” An isolated nucleic acid or protein can exist in substantially purified form, or can exist in a non-native environment such as, for example, a host cell. Unless it is particularly specified otherwise herein, the proteins, virion complexes, antibodies and other biological molecules forming the subject matter of the present invention are isolated, or can be isolated.

The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to a reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and “or” and similar referents in the context of describing the invention are to be construed to cover both the singular and the plural, unless otherwise indicated herein or clearly contradicted by context. Thus, for example, reference to “a subject polypeptide” includes a plurality of such polypeptides and reference to “the agent” includes reference to one or more agents and equivalents thereof known to those skilled in the art, and so forth.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventor for carrying out the invention. Variations of those embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention. Further, all publications mentioned herein are incorporated by reference in their entireties.

The invention will now be illustrated by the following non-limiting Examples.

### Example 1

Screening of Cultivars for Tissue Culture Response and Establishment of Genetic Transformation in a High-Yielding and Disease-Resistant Cultivar of Theobroma cacao

### Abstract

A highly efficient transformation protocol is a prerequisite to developing genetically modified and genome-edited crops. A tissue culture system spanning culture initiation from floral material to conversion of embryos to plants has been tested and improved in Theobroma cacao. Nine cultivars were screened for their tissue culture response and susceptibility to Agrobacterium transfer DNA delivery as measured through transient expression. These key factors were used to determine the genetic transformability of various cultivars. The high-yielding, disease-resistant cultivar INIAPG-038 was selected for stable transformation and the method was further optimized. Multiple transgenic events were produced using two vectors containing both yellow fluorescent protein and neomycin phosphotransferase II genes. A two-fold strategy to improve both T-DNA delivery and secondary somatic embryogenesis rates was conducted to improve overall transformation frequency. The use of Agrobacterium strain AGL1 and cotyledon tissue derived from secondary somatic embryos ranging in size between 4 to 10 mm resulted in the highest T-DNA delivery efficiency. Furthermore, the use of higher concentrations of basal salts and cupric sulfate in the medium increased the frequency of explants producing greater than ten embryos by five-fold and four-fold during secondary somatic embryogenesis, respectively. Consequently, an optimal combination of all these components resulted in a successful transformation of INIAPG-038 with 3.7% frequency at the T₀ plant level. Grafting transgenic scions with undeveloped roots to non-transgenic seedlings with healthy roots helped make plantlets survive and facilitated quick transplantation to the soil. The presented strategy can be applied to improve tissue culture response and transformation frequency in other Theobroma cacao cultivars.

### Introduction

Theobroma cacao L. is an economically important crop grown predominantly in Africa, although the species was originally domesticated in South America where significant production still remains. Despite Theobroma cacao beans being a commodity in a multibillion-dollar industry, there have been severe losses from disease to both crop yield and tree survival. Obstacles such as long juvenile period, issues with heterozygous and heterogeneous populations, long-life cycle and funding constraints on conventional Theobroma cacao breeding programs make desired traits like high yield, disease resistance and bean quality difficult to combine into a single cultivar. However, both genome editing and transgenic approaches can be used to improve and greatly accelerate the pace of trait development.

The first stable transformation to regenerate Theobroma cacao plants was reported in 2003 and since then there have been only a few other successful reports of the transformation of TcChi1, TeLEC2 and D5C and pD4e1 into Theobroma cacao. Transient expressions of CRISPR/Cas9 was also used to partially knockout TcNPR3 to improve defense response in Theobroma cacao. The introduction of BABY BOOM transcription factor into Theobroma cacao significantly increased transformation frequency, but it caused a regeneration issue with an abnormal phenotype. These previously mentioned experiments were performed on the amenable cultivar PSU SCA-6. Previous to this study, all reports published in the literature of transgenic plants being recovered were only in cultivar PSU SCA-6. Although PSU SCA-6 is not identical to SCA-6, the original SCA-6 was collected from the wild in the Peruvian Amazon and has some natural disease resistance. However, this cultivar is not highly productive. For genetic transformation to be used most effectively, the ability to transform many Theobroma cacao cultivars, including those less-amenable to transformation, must be established. Ideally, a single genotype-independent transformation method would be developed, but similar to somatic embryogenesis which has had broad success across the plant kingdom, there still exists a high degree of genotype-to-genotype variation and so protocol customization is necessary. Optimization of the basal salts, duration of hormone treatments, explant quality, and explant type are effective ways to improve the culturing response of recalcitrant cultivars.

Several explant types are used in Theobroma cacao tissue culture and the transformation process, which include petals and staminodes from unopened, immature flowers, cotyledon tissue from somatic embryos, and intact somatic embryos. Petals and staminodes are capable of primary somatic embryogenesis and are the starting point for all subsequent processes. Cotyledon tissue derived from somatic embryos is an alternative tissue type and efficient at secondary, tertiary and quaternary somatic embryogenic responses. Mature somatic embryos undergo conversion to plantlets and are able to be transplanted to soil. So far, all previous transformation methods used an embryogenic approach utilizing cotyledon tissue from somatic embryos. The advantages to this approach include previously described methodologies, the predominant single-cell origin of embryos, and the high number of embryos cotyledon tissue are capable of producing. Alternatively, an organogenic approach was not thoroughly explored because of the lack of previously described methods in Theobroma cacao, the apparent difficulty of establishing a new tissue culture system, and the non-clonal nature of seed which would result in the loss of the original cultivar if used as an organogenic donor material.

In this study we report a successful Agrobacterium-mediated transformation and regeneration protocol for INIAPG-038, using improved somatic embryogenesis and Agrobacterium infection. INIAPG-038 is a breeding line developed by Mars Wrigley, Incorporated in collaboration with United States Department of Agriculture (USDA) and Instituto Nacional de Investigaciones Agropecuarias (INIAP). It is a highly productive cultivar which has tolerance to fungal diseases such as frosty pod disease and witches broom disease. We also demonstrate a method for evaluating how amenable a cultivar is to tissue culture and transformation as well as provide data on the cultivars we screened.

### Materials and Methods

### Plant Material

Nine Theobroma cacao cultivars, SCA-6, CCN-51, INIAPG-038, INIAPT-374, INIAPG-152, INIAPM-053, INIAPG-029, INIAPT-484, and Matina 1-6, were used for tissue culture response experiments. Unopened, immature flowers originated from USDA Agricultural Research Service Subtropical Horticulture Research Station in Miami, FL where they were harvested from the greenhouse in the morning then shipped overnight to the Innovative Genomics Institute (IGI) Plant Genomics and Transformation Facility, Berkeley, CA. Cultivars were primarily chosen based on their consistent availability of flowers. The flowers were sealed within a 50 mL Falcon conical centrifuge tube which contained a 5 mM dithiothreitol solution and shipped in a Styrofoam insulated box along with ice packs to maintain a temperature of approximately 4° C.

### Initiation of Primary Embryogenesis Using Flower Material and Improvements

Immature flowers were used to initiate primary somatic embryos according to the previous protocols (Li Z, Traore A, Maximova S N, Guiltinan M J (1998) Somatic embryogenesis and plant regeneration from floral explants of cacao (Theobroma cacao L.) using thidiazuron. In Vitro Cell Dev Biol—Plant 34:293-299; Garcia C, Correa F, Findley S, Almeida A A, Costa M, Motamayor J C, Ray S, Marelli J P (2016) Optimization of somatic embryogenesis procedure for commercial clones of Theobroma cacao L. Afr J Biotechnol 15:1936-1951 (“Garcia et al. 2016”)) with some modifications. The medium composition followed was that of Garcia et al. 2016. Explants of 9 cultivars were observed after 3 to 4 mo of culture to determine the rate at which both staminode and petal explants formed embryos; a brief summary of the method follows. To initiate flower material it is surface-sterilized using a 20% (v/v) bleach solution (final concentration of 1.05% sodium hypochlorite) containing 0.002% Tween 20 then dissected, the petals and staminodes are placed onto primary callus growth (PCG) medium for 2 wk then transferred to secondary callus growth (SCG) medium (Garcia et al. 2016) for two wk, after which it is transferred to ED4 (embryo development medium containing 4% sucrose, Garcia et al. 2016) for two wk then subsequently on ED3 (embryo development medium containing 3% sucrose, Garcia et al. 2016) were it remains until embryos have formed and observations made. The total amount of staminodes and petals used for each cultivar as well as the number of replicates for each cultivar varied, they are as follows: SCA-6 with 1879 total explants across 2 replicates, CCN-51 with 985 total explants across 6 replicates, INIAPG-038 with 716 total explants across 7 replicates, INIAPT-374 with 623 total explants across 4 replicates, INIAPG-152 with 395 total explants across 4 replicates, INIAPM-053 with 295 total explants across at 4 replicates, INIAPG-029 with 260 total explants across 4 replicates, INIAPT-484 with 281 total explants across at 4 replicates, and Matina 1-6 with 645 total explants across 4 replicates.

### Initiation of Secondary Somatic Embryogenesis Using Primary Embryos

To induce secondary somatic embryogenesis, cotyledons were excised from primary somatic embryos and dissected into 4 to 10 mm segments. These explants were handled according to the method described by Garcia et al. 2016, although the SCG medium used was from the later. Secondary somatic embryos were harvested from these cultures while they were on ED3 and used for conversion to plantlets and Agrobacterium transformation. Initially, four cultivars, INIAPG-038, CCN-51, SCA-6, and INIAPT-374, were screened for the rates at which primary somatic embryo explants formed secondary somatic embryos. The embryos yielded from these explants were counted at approximately 10 wk and each explant ranked into one of four ratings: no embryos produced, 1 to 4 embryos produced, 5 to 9 embryos produced, and ≥10 embryos produced. The total amount of explants used for each cultivar as well as the number of replicates for each cultivar varied, were as follows: INIAG-038 with 2698 explants across 9 replicates, CCN-51 with 791 explants across 7 replicates, SCA-6 with 2514 explants across 8 replicates, and INIAT-374 with 752 explants across 5 replicates.

### Embryo Conversion and Plantlet Regeneration

Somatic embryos remained on ED3 medium until they were approximately 15 to 20 mm in length, at which point they were ready for conversion to plantlets. The conversion process began when these somatic embryos were placed onto ED6 medium (embryo development containing 6% sucrose, Garcia et al. 2016) and were exposed to low levels of light-emitting diode (LED) light, 10 to 30 μmol m-² s-¹. These embryos were incubated at 27° C. and the Petri dishes were then wrapped with 3M Micropore tape (3M, St. Paul, MN) to allow for gas exchange. After two wk on ED6 the embryos were transferred to embryo development-light (EDL) media and then maintained on EDL with transfers every two weeks. As the cultures developed, they were slowly exposed to higher LED light intensities, 30 to 60 μmol m-² s-¹. The conversion rates of various cultivars were measured and compared. An embryo was considered converted if it produced a shoot with a meristem and a root at least 1 cm in length. When the plantlets were 2.5 cm tall, they were transferred to a Phytatray™ II (Sigma-Aldrich, St. Louis, MO) containing 100 mL of medium and sealed with 3M Micropore tape. The cultivars tested include INIAPT-374, CCN-51 and INIAPG-038. The total amount of embryos used for each cultivar as well as the number of replicates for each cultivar varied, were as follows: T-374 with 256 embryos across 4 replicates, CCN-51 with 227 embryos across 8 replicates, and INIAPG-038 with 868 embryos across 9 replicates.

### Transfer-DNA (T-DNA) Delivery Efficiency Test

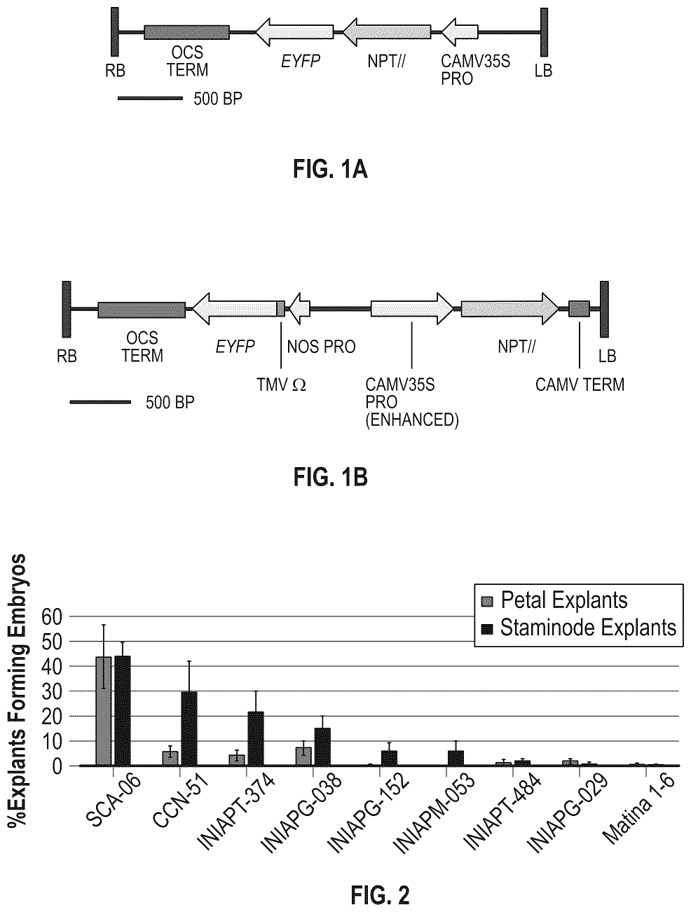

Four cultivars, INIAPG-038, SCA-6, CCN-51, and INIAPT-374 were tested for transient fluorescent protein expression (FPE) after transformation. Two different binary vectors, pDDNPTYFP-1 and pDDNPTYFP-2, were used in Theobroma cacao transformation experiments (FIGS. 1A-1B); pDDNPTYFP-1 contains neomycin phosphotransferase II (nptII)-GlyLink-yellow fluorescent protein (YFP) translational fusion driven by the cauliflower mosaic virus 35S (CaMV35S) promoter (FIG. 1A) while pDDNPTYFP-2 contains two separate gene cassettes, nptII driven by the enhanced CaMV35S promoter and YFP driven by the Nos promoter/TMV Ω enhancer (FIG. 1B). Both vectors were built in the pCAMBIA2300 backbone. Secondary embryos of similar size, color and morphology were selected from each cultivar and infected using the methods later described using the Agrobacterium strain AGL1. Explants were assessed based on FPE 7 d after Agrobacterium infection. Each explant was examined and ranked based on the percentage of its surface area that is expressing YFP. There were three rankings in total: 0 to 5% FPE coverage, 6 to 20% FPE coverage, and >20% FPE coverage. The treatment with the greatest percent of explants with >20% FPE coverage was selected as the best, however, it was sometimes useful to consider the distribution throughout all the rankings. The total amount of explants used for each cultivar as well as the number of replicates for each cultivar varied, were as follows: INIAPG-038 with 2025 explants across 16 replicates, CCN-51 with 472 explants across 6 replicates, SCA-6 with 2905 explants across ten replicates, and INIAPT-374 with 449 explants across 6 replicates.

### Transformation and Tissue Culture Improvements in INIAPG-038

Four Agrobacterium strains, LBA4404, AGL1, EHA105 and GV3101, containing pDDNPTYFP-1 were tested for T-DNA delivery efficiency. Three separate replicates comparing all 4 strains were conducted, the total explants used for each treatment of each replicate ranged from 130 to 200 and all material was sourced from INIAPG-038. All excised cotyledon tissue from the secondary somatic embryos was collected into a single Petri dish and randomized before being separated and treated with four different Agrobacterium strains. Prior to infection, each Agrobacterium strain was suspended into the infection medium and then diluted so that each Agrobacterium treatment was the same optical density at 600 nm wavelength (OD₆₀₀ₙₘ). These experiments were evaluated based on FPE coverage at 1 wk after infection using the method described previously.

The effect of secondary somatic embryo size on T-DNA delivery was also examined in INIAPG-038. Somatic embryos ranging in size between 4 to 10 mm were compared to somatic embryos 10 to 20 mm. The total amount of explants used and number of replicates for each treatment are the following: 4 to 10 mm embryo size used 1007 explants across 9 replicates, and the 10 to 20 mm embryo size used 714 explants across 5 replicates. Explants were evaluated with the method described previously.

The effect of doubled woody plant medium (WPM) basal salt concentration as well as WPM adjusted to 5.0 μM of CuSO₄ in SCG was examined for their effect on secondary embryo production in INIAPG-038. The treatments, total number of explants and replicates are as follows: Standard 1×SCG medium evaluated with 198 explants across 3 replicates, 1×SCG containing additional CuSO₄ evaluated with 327 explants across 5 replicates, and 2×SCG containing double concentration of WPM basal salts evaluated with 219 explants across 5 replicates. The explants were evaluated with the method described previously.

### Stable Transformation of INIAPG-038

### Agrobacterium Infection, Co-Cultivation, Embryogenesis, and Conversion

Transformations were performed using cotyledon tissue derived from somatic embryos of 4 to 10 mm length, white or tan in color and with normal morphology. The donor material used for the transformation process was secondary somatic embryos, making the transformation a tertiary somatic embryogenic process. To prepare the explants, cotyledon tissue from the previously selected secondary embryos was excised then dissected into 4 to 10 mm segments. The infection step was performed by suspending the explants in 10 to 20 mL of liquid medium harboring Agrobacterium contained within a sealed 50 mL test tube. The infection medium consisted of MS basal salt and vitamins containing 200 μM acetosyringone, 1 mg L⁻¹ indoleacetic acid and 2 mg L⁻¹ zeatin. The Agrobacterium was thoroughly suspended in the liquid infection medium and the OD₆₀₀ₙₘ was measured prior to use and adjusted to 0.400. After 1 to 2-h of agitation, the Agrobacterium infected cotyledon explants were transferred to a SCG based co-cultivation medium and incubated at 21° C. for 92±8-h before the Agrobacterium was washed away. To remove the Agrobacterium the explants were transferred from cocultivation medium into a 50 mL Falcon conical centrifuge tube containing 5 mL of liquid SCG medium and agitated using a 1 mL pipette for 1 min, then the liquid SCG medium was decanted and additional 20 mL of liquid SCG medium was added and the tube was agitated for 1 hr using an orbital shaker at 75 rpm. The cotyledon tissue was then transferred to a modified SCG medium containing 200 mg L⁻¹ cefotaxime and 200 mg L⁻¹ timentin. The tissue remained on this antibiotic containing SCG for between 2 and 4 wk before transfer to ED4 medium containing the same antibiotics for another 2 wk afterwards the tissue was maintained on ED3 with antibiotics. Antibiotics remained in the medium whenever there were explants that had been directly exposed to Agrobacterium. However, later in the process during the conversion of quaternary somatic embryos the antibiotics were removed from the medium. The first, second and third transformations were conducted without the use of chemical selection and transgenic embryos were identified using visual marker selection. The fourth transformation included the use of 100 mg L⁻¹ of kanamycin for chemical selection. After two wk of resting on SCG containing timentin and cefotaxime but without kanamycin the explants were moved to ED4 containing both antibiotics and kanamycin. This kanamycin concentration was maintained for 1 mo then increased to 150 mg L⁻¹ while the explants were on ED3.

The Agrobacterium-infected tissue underwent indirect somatic embryogenesis and was observed using a fluorescent microscope multiple times between one and four mo during the formation of embryos. After the identification of a transgenic event through the use of the fluorescent microscope the transgenic, tertiary somatic embryos (TSEs) were removed from the remainder of the non-transgenic tissue and isolated on ED3. To generate multiple quaternary somatic embryos (QSEs) from a single transgenic TSE an additional somatic embryogenesis step was used. This additional somatic embryogenesis step follows the same method for generating secondary somatic embryos, cotyledons are dissected and placed onto SCG for 2 wk then ED4 for 2 wk then maintained on ED3. QSE conversion and plantlet regeneration were conducted as described above.

### PCR Analysis

A CTAB DNA extraction protocol was used to extract genomic DNA from leaf materials of non-transgenic INIAPG-038 and transgenic INIAPG-038 plants. To test the presence of nptII in transgenic plants, the primer set, NPTII 3F (5′-CAAGATGGATTGCACGCAGGTT-3′ (SEQ ID NO: 1)) and NPTII 4R (5′-TAGAAGGCGATGCGCTGCGAAT-3′ (SEQ ID NO:2)) was used while the presence of eyfp was determined using the primer set, EYFP 3F (5′-TAAACGGCCACAAGTTCAGCG-3′ (SEQ ID NO:3)) and EYFP 4R (5′-AGGACCATGTGATCGCGCTTC-3′ (SEQ ID NO:4)). To test for the possible presence of Agrobacterium contamination in the extracted DNA samples or a read-through event three genes on the Agrobacterium backbone were also tested for. The presence of pVS1 was tested for using the primer set PVS1 1F (5′-ATGAAGGTTATCGCTGTACT-3′ (SEQ ID NO:5)) and PVS1 2R (5′-CTGATTCAAGAACGGTTGTG-3′ (SEQ ID NO:6)) while the presence of npt1 was tested for using the primer set NPT1 1F (5′-CTCCTGCTAAGGTATATAAGC-3′ (SEQ ID NO:7)) and NPT1 2R (5′-AATCAGGCTTGATCCCCAGT-3′ (SEQ ID NO:8)). For each PCR reaction mixture, the following reagents were used: 25 μL Dream Taq PCR Master Mix (2×) (Thermo Fisher Scientific, Grand Island, NY), 1.0 μL forward primer at 10 μM, 1.0 μL reverse primer at 10 μM, 21 μL H₂O, and 2.0 μL genomic DNA at approximately 100 ng μL⁻¹, for a total volume of 50 μL. The PCR was carried out with the following thermal cycler programming: hold at 95° C. for 3 min, 16 cycles of 94° C. for 30 sec, 58° C. for 30 sec (−0.5° C./cycle), 68° C. for 1 min, 50 cycles of 94° C. for 30 sec, 54° C. for 30 sec, 69° C. for 1 min; and then elongation at 68° C. for 7 min, and remained at 4° C. for infinite. For each PCR reaction, 20 μL were loaded onto a 0.8% agarose gel for electrophoresis.

### Fluorescent Visualization

Fluorescent images of embryos and plantlets of transgenic INIAPG-038 events were visualized with a fluorescent Leica M165 FC stereomicroscope, equipped with Leica DFC7000 T (JH Technologies, Fremont, CA); using two microscopic filters, brightfield and ET YFP with 514 nm excitation and 527 nm emission. The microscope is linked to a camera imaging software, Leica Application Suite version 4.9, which was used to capture the fluorescent images. Screening of fluorescent activity was measured at 30× magnification.

### Transplantation to Soil and Acclimatization

After embryo conversion while the plantlets are 10 to 13 cm in height and have six to ten leaves they were transferred to soil. The plantlets were transferred into a well-draining soil, mixing equal parts Sun Gro™ potting mix (Sun Gro Horticulture Distribution Inc., Agawam, MA) and perlite was found to work well. When transplanting the agar was removed by hand and by dipping the roots into water. The plantlets were then planted into square 4-inch pots with pre-moistened soil mix and immediately placed in a 1020 water tray (Greenhouse Megastore, West Sacramento, CA) then covered with a 7 inch vented humidity dome (Greenhouse Megastore, West Sacramento, CA) with all vents closed. On top of the humidity dome shades were placed to reduce the light intensity to 60 μmol m-² s-¹. The tray, humidity dome, shades and plants were kept inside a Conviron plant growth chamber with the parameters described later. Over the course of one wk the vents on the humidity dome were opened and the shades were removed. After this acclimatization process the plants were able to survive in the growth chamber with an ambient environment of 27° C., 60 to 70% RH and 120 to 180 μmol m-² s-¹ light intensity.

Grafting of Transgenic Scions onto In Vitro Germinated Rootstock

Wedge-shaped grafting was done as follows. Theobroma cacao pods of an unspecified Amelonado group (GRIN accession #PI 668412) were surface sterilized using 10% (v/v) bleach solution (final concentration of 0.525% sodium hypochlorite) for 15 min, then the pod was cut open and the seeds removed. The thick pulp was then removed from the seeds. The seeds were further surface sterilized using 10% (v/v) bleach solution (final concentration of 0.525% sodium hypochlorite) for 15 min. The sterilized seeds were then soaked in sterile water for 72 h. Afterwards, these pre-germinated seeds were planted into autoclave-sterilized peat pellets (Jiffy Products of America Inc. Lorain, OH) soaked in a liquid EDL medium. The seedlings were kept in Phytatrays™ and cultured similar to tissue culture-derived plantlets.

When both a transgenic scion with poor roots and a non-transgenic in vitro root stock with strong, healthy roots were available, grafting was performed using a “V” shape cut similar to a saddle or cleft graft on the seedling and two diagonal cuts exposing the cambium layer of the transgenic scion. The scion and rootstock were then combined and secured using both a paper clip and a 1 cm segment of a polypropylene straw cut down the side to resemble a “C”. All the cuts were performed and secured using sterilized tools and materials. The grafts were then sealed in Phytatrays™ with micropore tape and liquid EDL medium was periodically added to maintain proper moisture. After the scion and rootstock fused, they were transplanted to the soil using the previously outlined method.

### Results and Discussion

To establish a successful, highly efficient transformation protocol, a tissue culture system spanning the initiation of floral material to the regeneration of plantlets into soil has been tested and improved in Theobroma cacao. Multiple cultivars were screened for their response to (1) primary embryo induction from floral material, (2) secondary embryo production from cotyledon tissue derived from primary somatic embryos, (3) embryo conversion to plantlets, and (4) T-DNA delivery efficiency via Agrobacterium. These four factors were used to determine how amenable various cultivars would be for genetic transformation.

### Tissue Culture Response and INIAPG-038 Improvements

Petals and staminodes were used to initially test somatic embryogenesis in nine Theobroma cacao cultivars. Staminode explants generally formed embryos at a higher frequency than petal explants in all cultivars except SCA-6 where they were equal. (FIG. 2). The cultivar with the highest percentage of floral explants forming embryos was SCA-6 with 44% of petals and 44% of staminodes forming embryos (FIG. 2). The next best performing cultivars were CCN-51 with 6% of petals and 29% of staminodes, and INIAPT-374 with 4% of petals and 21% of staminodes and INIAPG-038 with 7% of petals and 15% of staminodes producing embryos at their respective rates. The remainder of the cultivars had less than 10% of floral explants producing embryos with INIAPG-152 and INIAPM-053 performing slightly better than the more recalcitrant INIAPT-484, INIAPG-029 and Matina 1-6.

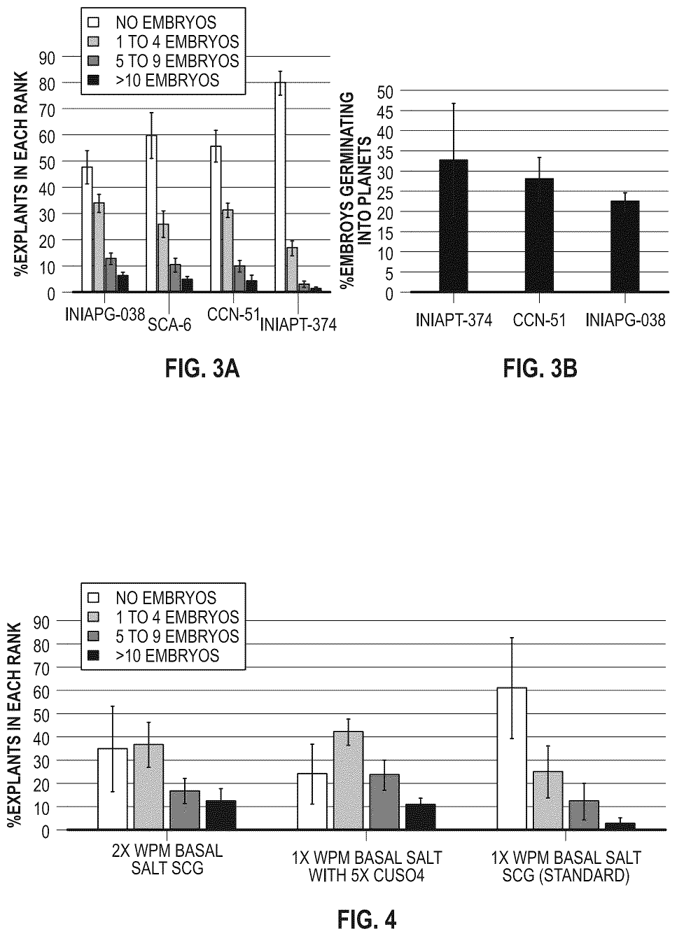

The four cultivars, SCA-6, CCN-51, INIAPT-374 and INIAPG-038, showing the best primary somatic embryo formation (FIG. 2) were further tested for their secondary somatic embryo production (FIG. 3A). It was observed that INIAPG-038 had the highest percentage of explants producing greater than 10 embryos at 6.2%. It also had the greatest number of explants producing between 5 and 9 embryos at 12.6%. SCA-6 and CCN-51 had similar secondary embryo production to INIAPG-038 while INIAPT-374 was lowest in secondary embryo production with 1.0% and 2.8% percent of explants forming greater than 10 embryos and percent of explants forming between 5 and 9 embryos, respectively. In total three cultivars were tested for the rate at which embryos would regenerate into plantlets (FIG. 3B). INIAPT-374 had embryo regeneration rates of 33%. CCN-51 and INIAPG-038 embryos regenerated to plantlets at a rate of 28% and 22%, respectively. However, there was no significant difference among these 3 cultivars.

With regard to experiments conducted to improve secondary somatic embryogenesis in INIAPG-038, it was observed that using a 2-fold concentration of WPM basal salts or WPM adjusted to 5.0 μM of CuSO₄ in SCG performed better than the standard SCG medium containing 1-fold WPM basal salts (FIG. 4). The standard SCG had 2.5% of explants forming greater than 10 embryos and 12.0% forming between 5 and 9 embryos, whereas 2-fold WPM basal medium resulted in 12.3% and 16.5%, respectively. The 2-fold WPM basal medium was significantly higher in percentage of explants forming greater than 10 embryos produced, compared to the standard SCG medium. The standard SCG medium, WPM-based medium, contains 1.0 μM CuSO4, 10-fold higher than MS medium. A five-fold copper level (5.0 μM) in SCG medium resulted in 10.9% of explants producing greater than 10 embryos and 23.4% of explants forming 5 to 9 embryos in INIAPG-038 (FIG. 4); it was significantly higher in percentage of explants forming greater than 10 embryos produced and significantly lower in percentage of explants forming no embryos produced, compared to the standard SCG medium This is consistent with the previous conclusion that the callus quality, callus growth and regenerability can be improved by increasing level of the micronutrient copper in barley and oat.

### T-DNA Delivery Efficiency Improvements and Optimizations

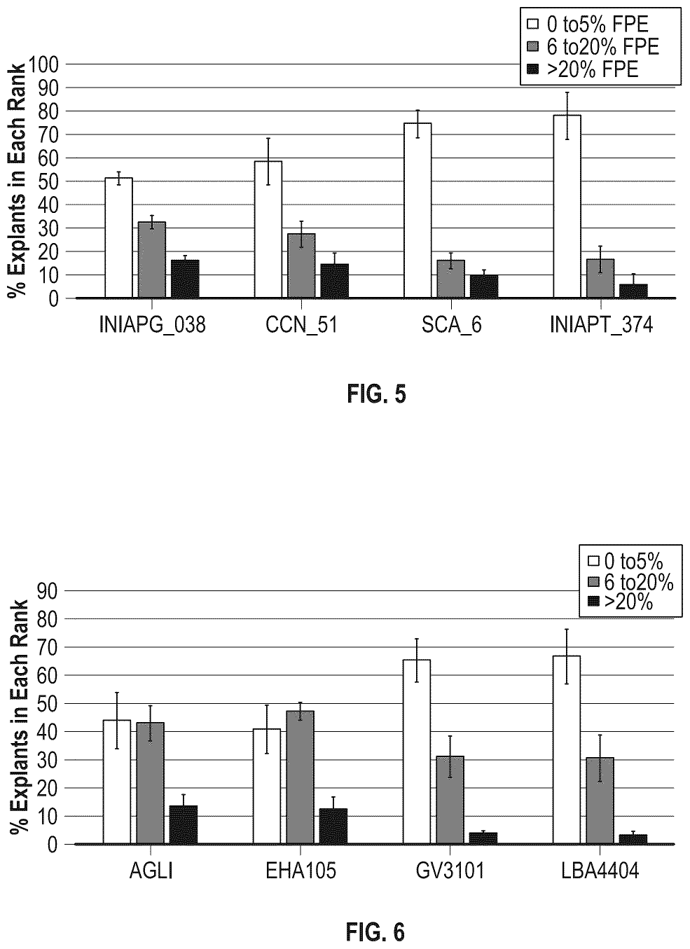

Experiments for the T-DNA delivery efficiency test were conducted using pDDNPTYFP-1 containing nptII::eyfp in the 4 cultivars with good tissue culture response: INIAPG-038, CCN-51, SCA-6 and INIAPT-374 (FIG. 5). Out of them, INIAPG-038 and CCN-51 had the T-DNA delivery efficiency with 16% and 14% of explants having >20% FPE coverage, respectively. The other 2 cultivars, SCA-6 with 9%, and INAPT-374 with 6% of explants ranked as >20% FPE coverage.

Of the four Agrobacterium strains tested, AGL1 and EHA105 produced the highest transient FPE coverage in INIAPG-038. Both LBA4404 and GV3101 performed poorly in T-DNA delivery efficiency (FIG. 6). The Agrobacterium strain AGL1 produced an average of 13% of explants ranked at >20% FPE, while EHA105 produced an average of 12%. GV3101 produced an average of 4% of explants ranked at >20% FPE, while LBA4404 produced an average of 3%. The Agrobacterium strain AGL1 producing a higher transformation frequency was previously observed in stable transformation of an elite maize inbred (Cho et al. 2014). However, other plant species or explants favored different Agrobacterium strains such as LBA4404 used in tobacco transformation and GV3101 used in tomato transformation.

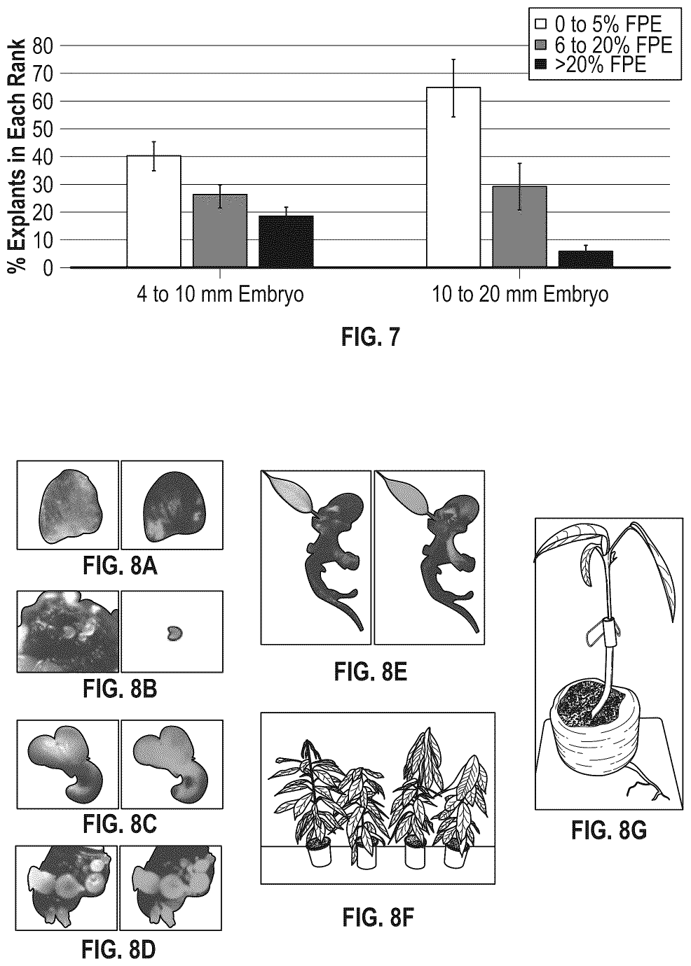

With regard to the INIAPG-038 explant type used for Theobroma cacao transformation, we observed that cotyledon tissue derived from primary or secondary somatic embryos had the highest transient FPE, while hypocotyl and radicle explants demonstrated very little transient FPE and embryo production after Agrobacterium infection (data not shown). Previously cotyledon tissues derived from mature embryos of PSU SCA-6 were used for transformation target. Our study showed that the embryo size of INIAPG-038 also affected transient FPE after transformation (FIG. 7). Cotyledon tissues derived from immature somatic embryos ranging in size between 4 to 10 mm outperformed those derived from mature embryos ranging in size between 10 to 20 mm. It was observed that embryos ranging in the 4 to 10 mm range produced explants with >20% FPE coverage at a rate of 19% whereas 10 to 20 mm embryos only produced explants with >20% FPE coverage at a rate 6%.

### Stable Transformation of INIAPG-038

INIAPG-038 was selected for further improvements to tissue culture response, T-DNA delivery efficiency, and eventually for stable transformation because it performed the best in both secondary somatic embryogenesis and T-DNA delivery. Although out of the 4 best cultivars from primary somatic embryogenesis screening it performed less efficiently in primary somatic embryogenesis (FIG. 2), this step is not as crucial for transformation and is not seen as a potential bottleneck. In addition, INIAPG-038 is a highly productive cultivar with tolerance to frosty pod disease and witches broom disease. Previous transformations were conducted on PSU SCA-6 because it is an amenable cultivar with high somatic embryogenesis and genetic transformation potential. This Forastero cultivar also demonstrates high production of primary somatic embryos when compared to Trinitario cultivars. Our decision to focus on INIAPG-038 was heavily weighed on the importance of the cultivar as well as the somatic embryogenic potential (FIGS. 2 and 3A) and the T-DNA delivery efficiency (FIG. 5).

Stable transformation experiments in INIAPG-038 were conducted by infecting cotyledon tissues derived from the secondary somatic embryos with AGL1 containing pDDNPTYFP-1 or pDDNPTYFP-2 (FIGS. 1A-1B). Six to ten cotyledon explants could be dissected from each embryo used in this process. Transient YFP expression was clearly observed 5 to 7 d after Agrobacterium infection (FIG. 8A). Five separate successful stable transformation experiments in INIAPG-038 were conducted by YFP visual marker selection. Kanamycin selection was not tight enough and non-transgenic embryo tissue still formed on the cotyledon explants even with a high level of kanamycin. In a method described earlier, 50 mg L⁻¹ of geneticin (G418) for nptII was used which greatly reduces the formation of non-transgenic tissues. Eight independent transformation events in the form of YFP-expressing globular embryos of INIAPG-038 were generated from the first 4 sets of experiments using cotyledon tissues derived from 82 somatic embryos; transformation frequency at the T₀ tissue level was 9.8% (8 out of 82) (Table 1, FIG. 8B). Eleven more independent transformation events were generated using 82 additional somatic embryos in the 5th set of experiments; transformation frequency at the T₀ tissue level was 13.4% (11 out of 82) (Table 1).

TABLE 1
Stable transformation frequencies of Theobroma cacao cv. INIAPG-038

| | | | | | | | Number of | | |
|---|---|---|---|---|---|---|---|---|---|
| Trans- | | | Number of | | | | quaternary | | |
| formation | | Number of | YFP- | T₀ tissue level | Number of | T₀ plant level | somatic | Number of | Number of |
| Experi- | | embryos | expressing | transformation | regenerable | transformation | embryos | plantlets | plantlets |
| ments | Vector used | dissected^{a} | eventy | frequency^{b} | events | frequency^{c} | produced | regenerated | in soil |
| Set 1 | pDDNPTYFP-1 | 32 | 2 | 6.3% | 1 | 3.1% | 68 | 15 | 11 |
| Set 2 | pDDNPTYFP-2 | 10 | 1 | 10.0% | 0 | 0.0% | 0 | 0 | 0 |
| Set 3 | pDDNPTYFP-2 | 24 | 3 | 12.5% | 1 | 4.2% | 16 | 4 | 3 |
| Set 4 | pDDNPTYFP-2 | 16 | 2 | 12.5% | 1 | 6.3% | 95 | 20 | 3 (1)^{d} |
| | Subtotal | 82 | 8 | 9.8% | 3 | 3.7% | 179 | 39 | 17 |
| Set 5 | pDDNPTYFP-2 | 82 | 11 | 13.4% | n.d. | n.d. | n.d. | o.d. | n.d. |
| | Total | 164 | 19 | 11.6% | | | | | |

^{a}After dissection a somatic embryo would produce 6 to 10 cotyledon explants
^{b}T₀ tissue level transformation frequency = (number of YPP-expressing events/number of embryos dissected) × 100%
^{c}T₀ plant level transformation frequency = (number of regenerable events/number of embryos dissected) × 100%
^{d}One out of 3 is a grafted plant and moved to soil to grow in the greenhouse
n.d.: not determined

The transformation process described herein yields only a single transgenic TSE (FIG. 8B) for every transformation event and the average rate of embryo conversion for INIAPG-038 was only 22.4% (FIG. 3B), so if one tried to regenerate transgenic TSEs directly, most transformation events would likely not be recovered as plants. To increase the probability of regenerating transformation events into plants an additional embryogenesis cycle was conducted to generate multiple transgenic QSEs from every transformation event prior to the embryo conversion step. Of the 8 transformation events generated from the first 4 sets of transformation experiments 3 transgenic TSEs developed into mature embryos (FIG. 8C) which were proliferated into multiple QSEs (FIG. 8D) and were capable of regeneration into plantlets (FIG. 8E), resulting in a T₀ plant level transformation frequency of 3.7% (3 out of 82) (Table 1). To transformation frequency was calculated as (# of regenerable events/# of embryos dissected)×100%. With quaternary somatic embryogenesis all three of the transgenic events, #1, 2 and 3, proliferated into 68, 16, and 95 total embryos, respectively (Table 1). One (event #3) of the three transgenic embryos was chimeric and non-transgenic QSEs were discarded based on YFP expression. Of the QSEs generated for each event 15, 4, and 20 of those embryos converted into plantlets, respectively (Table 1), giving an average of 21.8% rate of transgenic embryo conversion to plantlets. Of the embryos converted to plants for each event 11, 3, and 3 plants of each event were successfully acclimatized in soil, respectively (Table 1 and FIG. 8F).

Some regenerated transgenic INIAPG-038 plantlets had slow or poor development of roots and these could not survive in soil; therefore, grafting was attempted to resolve this issue. Plantlets with poorly developed root systems routinely die during transplanting to soil, there is also a risk of damaging well-established root systems during agar removal. However, grafting transgenic scions in vitro avoids these two issues since a robust well-rooted non-transgenic seedling is used and the entire intact root system within a sterilized Jiffy peat cube can be placed into the soil without disturbing the roots (FIG. 8G). In total, six transgenic scions with poor roots were grafted onto rootstock, four of these grafts survived. Out of them 1 plant of event #3 was transferred to soil to grow in the greenhouse (Table 1). In addition, grafting could facilitate the use of transgenic shoots that might otherwise not form roots. The use of grafting to overcome poor in vitro rooting and improve transformant recovery was also used previously in safflower and cotton.

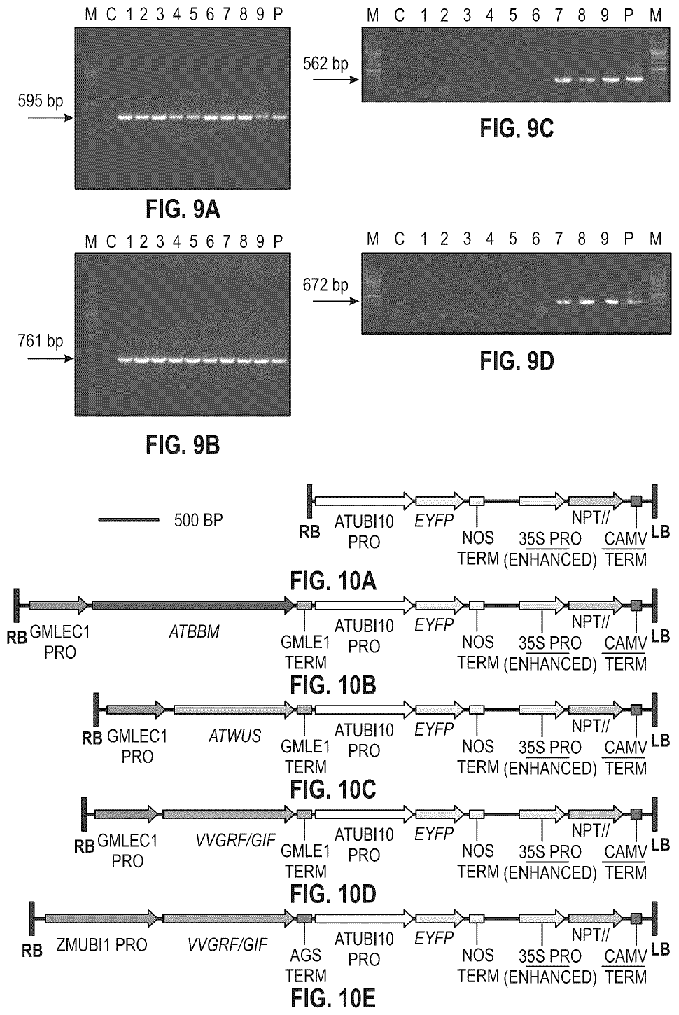

The presence of transgenes, eyfp and nptII, was confirmed by PCR analysis. The amplified product of 595 bp corresponding to the internal fragment of eyfp gene was observed from genomic DNA of all 9 tested transgenic plants derived from three independent events using eyfp gene-specific primers, EYFP 3F and EYFP 4R (FIG. 9A). An amplified fragment of 761 bp was also observed from all tested transgenic plants derived from three independent events using nptII gene-specific primers, NPTII 3F and NPTII 4R (FIG. 9B). No PCR-amplified eyfp and nptII fragments were detected in the non-transgenic control plant (FIGS. 9A, 9B) as expected. Additional PCR controls were performed using primers for the bacterial pVS1 origin of replication and nptII gene located outside the T-DNA borders to check the possibility of Agrobacterium contamination giving false-positive results. The absence of transgenes, pvs1 and npt1 was confirmed by PCR analysis in both the first and second events; however, the third event produced amplified products corresponding to two Agrobacterium vector backbone sequences (FIGS. 9C, 9D). Comparisons of the two Agrobacterium strains used for transformation as well as intact leaf samples from the third event were compared to determine if Agrobacterium contamination was the cause for all four genes being detected.

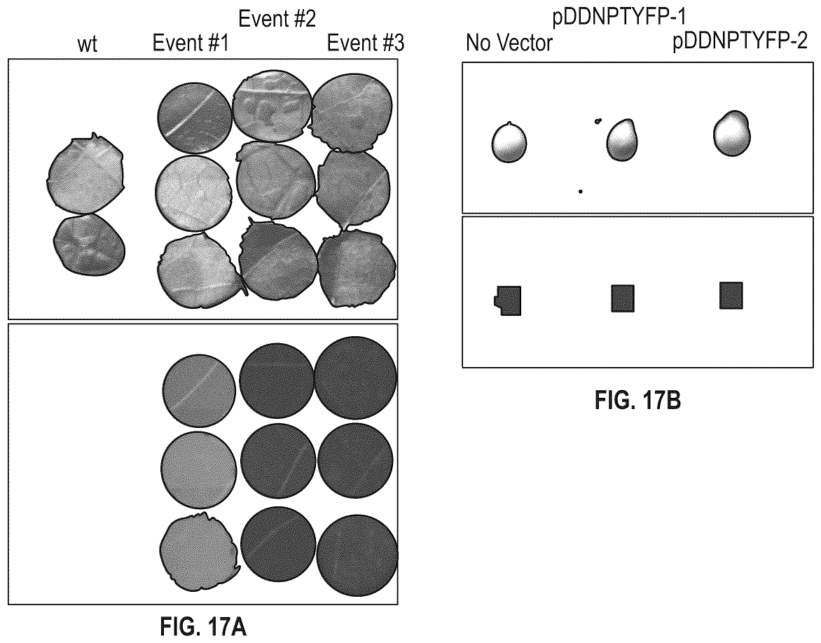

The Agrobacterium strains containing the eyfp gene cassette showed no YFP expression and the YFP expression of the leaf samples of the third event was uniform and consistent with that of the other two events (FIGS. 17A-17B). Stronger YFP expression driven by the CaMV35S promoter was observed in the leaf tissues compared to that driven by Nos promoter/TMV Ω enhancer. The presence of the Agrobacterium vector backbone sequences would then indicate that the third event was a read-through event and those genes are additionally present with the other transgenes. Read-through is integration into the plant genome of complete vector backbone sequences which could be the result of a conjugative transfer initiated at the right border and subsequent continued copying at the left and right borders. The ratio of the read-through plants in transgenic events ranges typically between 20% and 50% and is sometimes as high as 75% or more.

In conclusion, we have established a successful Agrobacterium-mediated transformation system for the production of transgenic Theobroma cacao plants using INIAPG-038, a high-yielding, disease-resistant cultivar. Previously only PSU SCA-6 has been reported for stable transformation. The results in this study can be applied to improve tissue culture response and transformation frequency in other Theobroma cacao cultivars.

### Example 2

Use of Transcription Factors to Improve Transformation Frequency and Reduce Genotype-Dependence in Theobroma cacao

### Vector Construction and Agrobacterium Strains

Morphogenic gene expression and visual marker cassettes were cloned into the pCAMBIA2300 CR3 binary vector (FIGS. 10A-10E). Initially, the A. thaliana ubiquitin-10 (AtUbi10) promoter, enhanced yellow fluorescent protein (eyfp) gene, and two A. tumefaciens nopaline synthase (Nos) terminators were PCR amplified using Phusion® High-Fidelity Polymerase [New England Biolabs (NEB)]. Purified amplicons were assembled into the KpnI-HF digested pCAMBIA2300 CR3 using the NEBuilder® HiFI DNA Assembly Cloning Kit (NEB). Next, the CR3 cassette was removed by overnight PmeI and KpnI-HFdigestion. DNA fragments were separated on a 0.75% TAE gel and the desired pCAMBIA2300 AtUbi10p:YFP fragment was extracted using the Monarch® DNA Gel Extraction Kit (NEB). For assembly of constructs bearing Le1p:AtBBM, Le1p:AtWUS, and Le1p:GRF/GIF, the following DNA elements were amplified and assembled with the linear pCAMBIA2300 AtUbi10p:eYFP fragment using the NEBuilder® HiFI DNA Assembly Cloning Kit. The seed-specific soybean lectin Le1 gene promoter (Le1p) and terminator were amplified from soybean genomic DNA. Baby boom (bbm) and Wuschel2 (wus) genes were amplified from A. thaliana genomic DNA. The chimeric GROWTH-REGULATING FACTOR (grf) and co-factor GRF-INTERACTING FACTOR (gif) gene was derived from Vitis vinifera (kindly provided by the Jorge Dubcovsky lab). For assembly of the construct bearing ZmUbi1p:GRF/GIF, the Zea mays ubiquitin-1 (Ubi1) gene promoter was amplified and assembled as described above. For assembly of AtUBQ10p:eYFP+enh35Sp:nptII, DNA ends of the PmeI/KpnI-digested pCAMBIA2300 AtUbi10p:eYFP fragment were blunted using T4 DNA polymerase (NEB) The blunt fragment ends were ligated by high concentration T4 DNA Ligase (NEB) to yield a circular plasmid. Each construct was transformed into electrocompetent cells of AGL1 by electroporation.

Transformation of Theobroma cacao

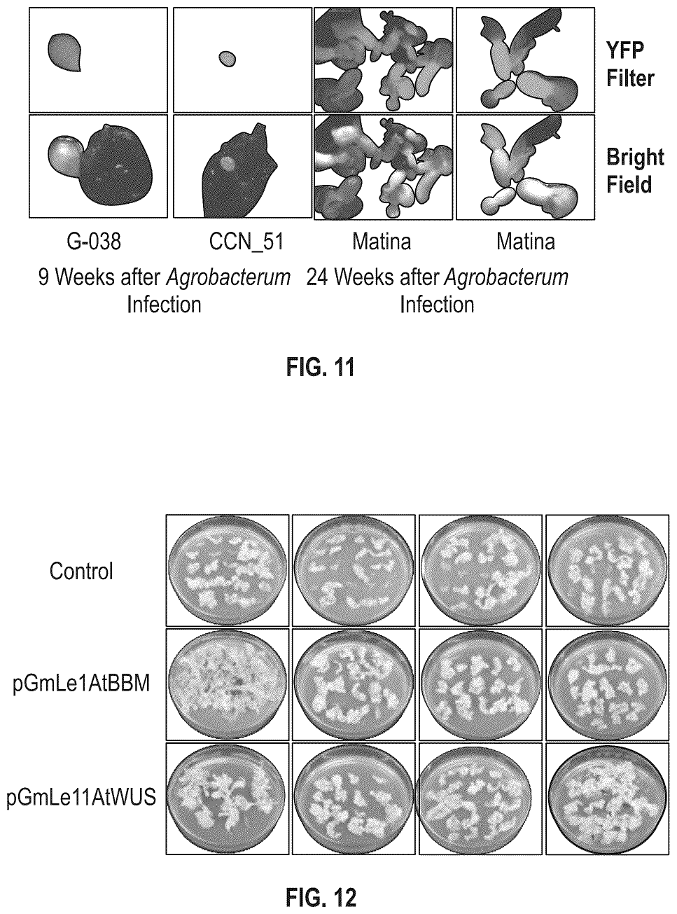

Cacao transformations were performed using cotyledon tissue derived from somatic embryos of 4 to 10 mm length, white or tan in color and with normal morphology, as described in Example #1. Five transformations conducted only with pGmLe1AtBBM (FIG. 10B). YFP-expressing embryos transformed with this vector could be detected 1-2 months after Agrobacterium infection compared to 3-4 months after Agrobacterium infection with the control vector. Thirty events were generated in all 3 cultivars tested, Matina, INIAPG-038 and CCN 51 (Table 2). Most these events were generated in Matina and 18 of these events have made it to the QSE stage. Based on early observations, most these events appear to be normal in morphology (FIG. 11). The abnormal embryo phenotype was observed in previous literature and yielded embryos incapable of regeneration when the constitutive CaMV35S promoter used. For each of the early Matina events there are at least a few embryos that have normal morphology and lack the tissue browning or greening that some of the material shows, so we expect most of the events will be capable of regeneration.

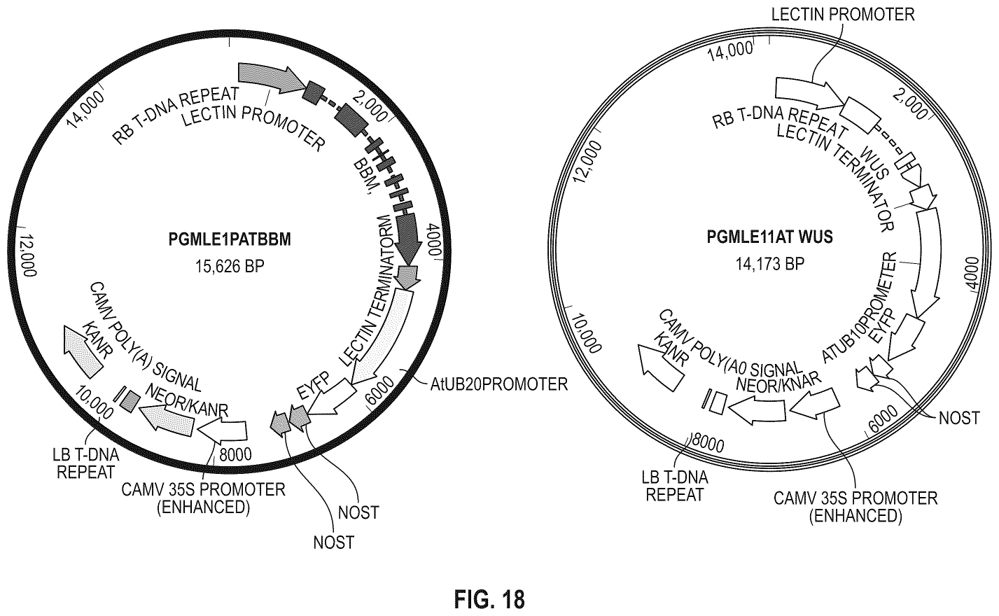

Two more sets of transformation experiments were conducted to compare the following 5 constructs on both Matina and INIAPG-038; control, pGmLe1AtBBM, pGmLe1AtWUS, pGmLe1VvGRF/GIF and pZmUbi1VvGRF/GIF (Table 2, FIGS. 10A-10E, FIG. 18). The first transformation yielded 1 event in Matina from pGmLe1AtBBM. A single event from both genotypes has progressed to the QSE stage from this experiment and the second one did 2 evens in INIAPG-038 (Table 2). There were no other events generated from any of the other constructs so far. The total donor material used for each construct was small, for Matina it averaged to 8.4 embryos per construct and for INIAPG-038 it was 14.8. The third and fourth transformations comparing all 5 constructs used 40 embryos of Matina per construct and 32 embryos of INIAPG-038 per construct (Table 2).

TABLE 2
Status of cacao transformation using transcription factor vectors

| | | | # | # Events | Events |
|---|---|---|---|---|---|
| | Donor | | Embryos | with YFP | with |
| Cultivar | Material | Construct | Infected | Expression | QSE |
| Matina | Secondary | pGmLe1AtBBM | 40 | 4 | 4 |
| | Embryo | | | | |
| G_038 | Secondary | pGmLe1AtBBM | 75 | 6 | 1 |
| | Embryo | | | | |
| Matina | Secondary | pGmLe1AtBBM | 75 | 13 | 11 |
| | Embryo | | | | |
| G_038 | Secondary | pGmLe1AtBBM | 75 | 5 | 1 |
| | Embryo | | | | |
| CCN_51 | Secondary | pGmLe1AtBBM | 75 | 2 | 1 |
| | Embryo | | | | |
| Matina | Secondary | control | 8.4 | 0 | 0 |
| | Embryo, | | | | |
| | SSE Culture | | | | |
| Matina | Secondary | pGmLe1AtBBM | 8.4 | 1 | 1 |
| | Embryo, | | | | |
| | SSE Culture | | | | |
| Matina | Secondary | pGmLe1AtWUS | 8.4 | 0 | 0 |
| | Embryo, | | | | |
| | SSE Culture | | | | |
| Matina | Secondary | pGmLelVvGRF/ | 8.4 | 0 | 0 |
| | Embryo, | GIF | | | |
| | SSE Culture | | | | |
| Matina | Secondary | pZmUbi1VvGRF/ | 8.4 | 0 | 0 |
| | Embryo, | GIF | | | |
| | SSE Culture | | | | |
| G_038 | Secondary | control | 14.8 | 0 | 0 |
| | Embryo, | | | | |
| | SSE Culture | | | | |
| G_038 | Secondary | pGmLe1AtBBM | 14.8 | 2 | 1 |
| | Embryo, | | | | |
| | SSE Culture | | | | |
| G_038 | Secondary | pGmLe1AtWUS | 14.8 | 0 | 0 |
| | Embryo, | | | | |
| | SSE Culture | | | | |
| G_038 | Secondary | pGmLe1VvGRF/ | 14.8 | 0 | 0 |
| | Embryo, | GIF | | | |
| | SSE Culture | | | | |
| G_038 | Secondary | pZmUbi1VvGRF/ | 14.8 | 0 | 0 |
| | Embryo, | GIF | | | |
| | SSE Culture | | | | |
| Matina | Secondary | control | 40 | nd | nd |
| | Embryo | | | | |
| Matina | Secondary | pGmLe1AtBBM | 40 | nd | nd |
| | Embryo | | | | |
| Matina | Secondary | pGmLe1AtWUS | 40 | nd | nd |
| | Embryo | | | | |
| Matina | Secondary | pGmLe1VvGRF/ | 40 | nd | nd |
| | Embryo | GIF | | | |
| Matina | Secondary | pZmUbi1VvGRF/ | 40 | nd | nd |
| | Embryo | GIF | | | |
| G_038 | Secondary | control | 32 | nd | nd |
| | Embryo | | | | |
| G_038 | Secondary | pGmLe1AtBBM | 32 | nd | nd |
| | Embryo | | | | |
| G_038 | Secondary | pGmLe1AtWUS | 32 | nd | nd |
| | Embryo | | | | |
| G_038 | Secondary | pGmLe1VvGRF/ | 32 | nd | nd |
| | Embryo | GIF | | | |
| G_038 | Secondary | pZmUbi1VvGRF/ | 32 | nd | nd |
| | Embryo | GIF | | | |

### Example 3

Use of Transcription Factor Genes for Transformation Test in Nicotiana tabacum and Nicotiana benthamiana
Transformation Protocol for Tobacco and Nicotiana benthamiana

Four to six weeks prior to transformation, seeds were sterilized with 20% bleach for 15 minutes, washed with sterile distilled H₂O four to five times, and were germinated in Phytatray™ II (Sigma-Aldrich, St. Louis, MO) containing tomato germination media (SIG). One day before transformation, a selected Agrobacterium strain containing a binary vector was streaked onto Agrobacterium-induction plates with 100 μM acetosyringone (AS) and appropriate antibiotic for selection. Infection buffer containing Agrobacterium was diluted in a 15 mL Falcon tube supplemented with 200 μM AS, 1 mg/L 6-benzylaminopurine (BAP), and 0.1 mg/L 1-naphthaleneacetic acid (NAA). An OD₆₀₀ reading of 0.400 (+/−0.015) of the infection buffer was used for transformation. Nicotiana tabacum (tobacco) or Nicotiana benthamiana explants were dissected by cutting off leaf tissue (5-10 mm²) and placing them on top of a sterile Whatman 70 mm (Cat. No. 1001-070) filter paper in a sterile 100 mm×25 mm petri dish, wet with 800 μL of the bacterial mixture. Tissues were infected by pipetting at least 5 mL of diluted bacteria mixture into petri dishes. The petri dish containing the leaf tissues were sealed with parafilm and were placed on an orbital shaker at room temperature for two hours. The Infection buffer was then removed, and the tissues were transferred onto to co-cultivation medium for three to four days of incubation at 21° C. Cultures were checked three days after co-cultivation to monitor excess Agrobacterium growth surrounding the tissues. The tissues were washed overnight in a 50 mL Falcon tube containing 15-mL infection medium with 200 mg/L cefotaxime, 200 mg/L timentin, 1 mg/L BAP, and 0.1 mg/L NAA if Agrobacterium overgrowth is observed. The leaf tissues that appeared to be free of bacterial overgrowth were washed for one more day and were blotted dry in a sterile filter paper for subsequent transfer onto the first round of selection. The tissues were transferred onto fresh selection media every two weeks maintaining contact with the media for efficient selection. Putative transgenic shoots that reached the height of at least 1.5 cm were transferred to rooting medium in a Phytatray™ II to generate the transgenic plants.

Effects of BABYBOOM (BBM) and WUSCHEL (WUS) on Transformation Frequency and Plant Phenotype in Transgenic Tobacco and N. benthamiana

Plant morphogenic genes such as BABY BOOM (bbm) and WUSCHEL (wus) were known to induce embryo-like structures or somatic embryos as well as enhance regeneration that led to the increase in transformation frequency of recalcitrant genotypes in corn. We assessed the effects of these two genes in dicot plant model systems tobacco and Nicotiana benthamiana.

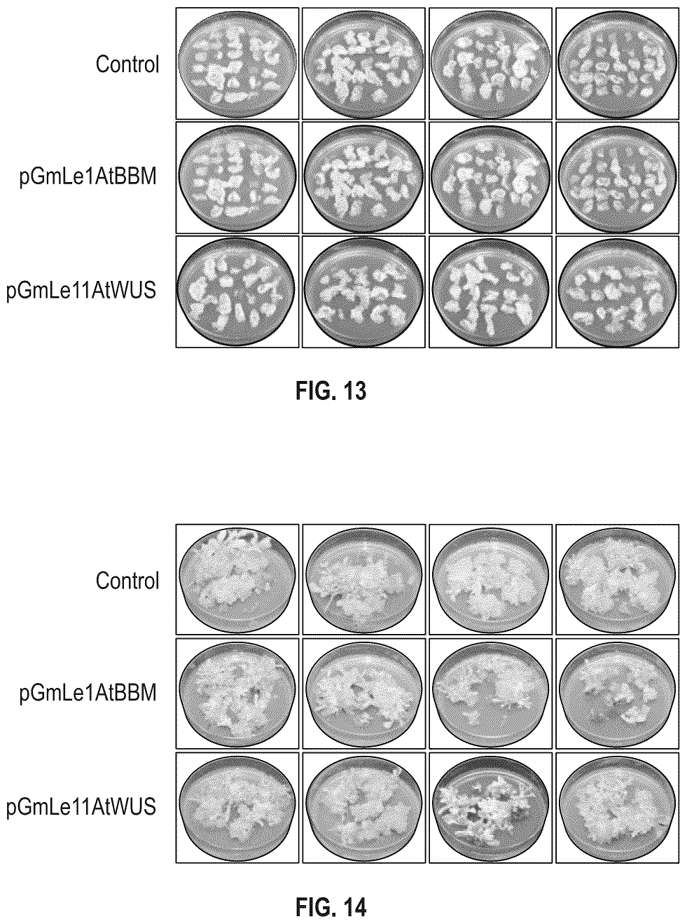

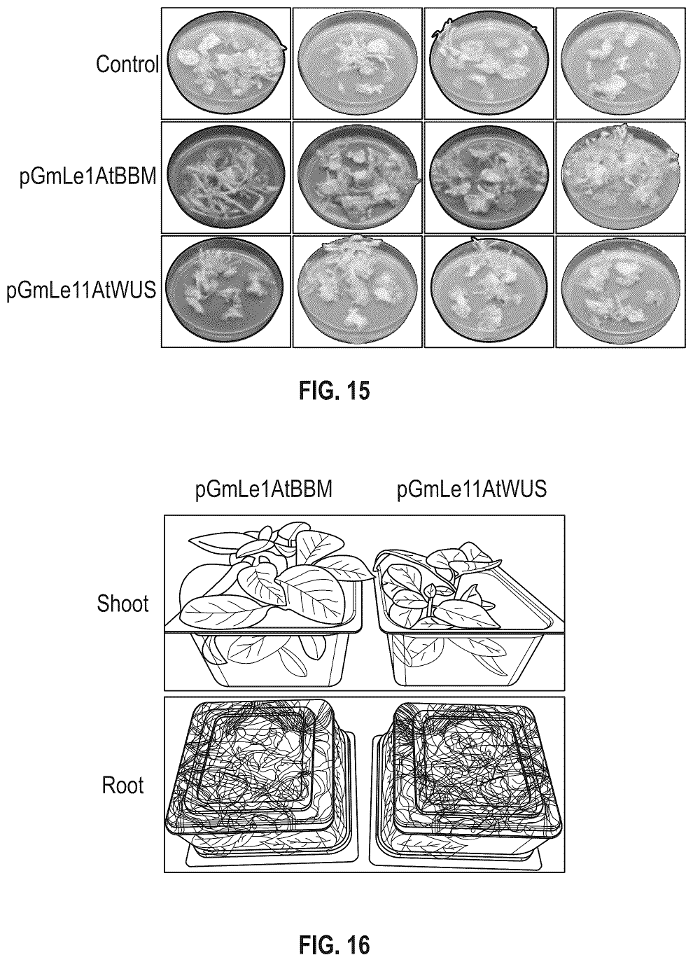

Based on the preliminary results we have obtained from the first set of transformation, it appears that there were differences observed between tobacco and N. benthamiana plants that were transformed with bbm and wus genes driven by the soybean embryo-specific lectin (Le1) promoter (Tables 3 & 4, FIGS. 12-15). After selecting the transformed leaf tissues in medium containing kanamycin during selection 30 days post infection, we observed profuse green sectors/small shoots developing in pGmLe1AtBBM putative transgenic tissues, followed by pGmLe1AtWUS, and the least green sectors were from the control construct NPTII-eYFP for both tobacco and N. benthamina. Here, we found that highest transformation efficiency can be observed in pGmLe1AtBBM>pGmLe1AtWUS>control. After 66 days in selection, putative transgenic tobacco plants were generated in pGmLe1AtBBM and lesser transgenic events in plants in pGmLe1WUS. However, N. benthamiana plants appear to have slower growth rate after selection where no plantlets (with root formation) were generated at the time when this data was recorded. The data includes qualitative assessment for both transformation efficiency and plantlet regeneration. Transformation efficiency using these transcription factor genes will be generated once the final putative transgenic plantlet counts will be completed. Currently, there are no significant phenotypic effects in the growth of shoots and root development of transgenic tobacco plants.

TABLE 3
Transformation efficiency and phenotypic effects in transgenic
tobacco plants at 66 days after Agrobacterium infection.

| | Transformation | | |
|---|---|---|---|
| Construct | Efficiency | Shoot Development | Root Growth |
| Control | ++ | − | − |
| pGmLe1AtBBM | ++++ | ++++ | ++++ |
| pGmLe1AtWUS | +++ | ++ | + |

− No plants with complete root and shoot regenerated as of data recording
+ Fair
++ Moderate
+++ High
++++ Highest

TABLE 4
Transformation efficiency and phenotypic effects in transgenic
Nicotiana benthamiana plants 66 days after Agrobacterium infection.

| Construct | Transformation Efficiency | Shoot Development |
|---|---|---|
| Control | ++ | + |
| pGmLe1AtBBM | ++++ | ++++ |
| pGmLe1AtWUS | +++ | ++ |

+ Fair
++ Moderate
+++ High
++++ Highest

### Example 4

### Use of Transcription Factor Genes for Transformation in Tomato

### Tomato Transformation

Solanum lycopersicum M82 seeds were surface sterilized in 20% (v/v) bleach solution containing one drop of Tween-20 for 15 min before rinsing three times with sterile autoclaved water. The seeds were germinated in Petri dishes containing 30 mL of Murashige and Skoog (MS) (Phytotech M524) based medium containing 2.15 g/l MS salts, 100 mg/l myo-inositol (Phytotech 1703), 10 g/l sucrose, 1 ml/l Gamborg's vitamin solution 1000× (Phytotech G219), 195.2 g/l MES, and 3.5 g/l Phytagel (Sigma-Aldrich P8169), pH 5.8. Cultures were maintained at 26° C. under 16 h light/8 h dark photoperiod at 57-65 μE m⁻² s⁻¹ for six to seven days.

One day prior to infection, Agrobacterium strains were streaked onto 20 mL Agrobacterium induction plates containing 5 g/l Bacto yeast extract (Gibco 288620), 10 g/l Bacto peptone (Gibco 211684), 5 g/l sodium chloride, 15 g/l Bacto agar (BD Difco 214050), 100 μM acetosyringone (AS) and 50 mg/l kanamycin and kept for 16 h at 28° C. Newly formed Agrobacterium were suspended in 5 mL liquid infection medium containing 4.33 g/l MS salts (Sigma-Aldrich M5519), 100 mg/l myo-inositol, 1 mL/l Gamborg's vitamin solution 1000× (Phytotech G219), 195.2 g/L MES, 30 g/l glucose (Sigma-Aldrich G7021), 200 μM AS, 2 mg/l trans-zeatin riboside, and 1 mg/l indole-3-acetic acid (IAA). Agrobacterium solutions were diluted to achieve an OD₆₀₀ of 0.400+/−0.015. M82 cotyledon tissues were excised from germinating seedlings and placed on sterile filter paper (Whatman 1001-070) containing 1 ml of Agrobacterium mixture. The tissues were placed abaxial side up and sliced to reach no longer than 5 mm in length. The remaining bacterial solution was transferred to the Petri dish and the tissues were placed on an 80 rpm incubating shaker at room temperature for two hours. The tissues were then transferred to co-cultivation medium adaxial side up containing 4.33 g/l MS salts, 100 mg/l myo-inositol, 1 ml/l Gamborg's vitamin solution 1000×, 195.2 g/l MES, 20 g/l glucose, 6 g/l agar (Sigma-Aldrich A1296), 200 μM AS, 2 mg/l trans-Zeatin riboside, and 1 mg/l IAA. The tissues were maintained in 21° C. for 96 h in the dark.

After 4 d, the tissues were transferred onto a clean filter paper with the addition of 800 ul liquid infection medium for 16-24 h at 21° C. The tissues were then transferred to the first round of selection medium containing 4.33 g/l MS salts, 100 mg/l myo-inositol, 1 ml/l Gamborg's vitamin solution 1000×, 195.2 g/L MES, 20 g/l sucrose, 8 g/l plant agar (Phytotech A296), 2 mg/l trans-zeatin riboside and 0.5 mg/l, 200 mg/l cefotaxime (Phytotech C380), 200 mg/l timentin (Phytotech T869), and 90 mg/l kanamycin (Phytotech K378). The tissues were maintained at 26° C. under 16 h light/8 h dark for two weeks before transferring onto the second round of selection medium containing 0.1 mg/L IAA. When shoots reached at least 3 mm tall, they were transferred to shoot elongation medium which contained 4.33 g/l MS salts, 100 mg/l myo-inositol, 20 g/l sucrose, and 8 g/l plant agar, 1 mg/l trans-zeatin riboside, 200 mg/l cefotaxime, 200 mg/l timentin, 1 ml/l Nitsch and Nitsch vitamins (1000×) and 90 mg/l kanamycin. After the shoots reached a height of 25 mm, they were transferred onto rooting medium containing 4.33 g/l MS salts, 30 g/l sucrose, 8 g/l plant agar, 0.2 mg/l IAA, 200 mg/l cefotaxime, 200 mg/l timentin, 1 ml/l Nitsch and Nitsch vitamins (1000×) and 60 mg/l kanamycin in Phytatrays (Sigma-Aldrich P5929).

All media were adjusted to a pH of 5.8 before autoclaving. Ingredients such as trans-zeatin riboside, IAA, kanamycin, cefotaxime, timentin, and Nitsch and Nitsch vitamins were filter-sterilized and added into autoclaved medium that was allowed to cool to 55° C.

### Effects of BABYBOOM (BBM) and WUSCHEL (WUS) on Transformation Frequency and Plant Phenotype in Transgenic Tomato

After selecting the transformed leaf tissues in medium containing kanamycin during selection 2.5 months post infection, the highest transformation frequency and fastest shoot development were observed from pGmLe1AtWUS. There was no difference in transformation frequency and shoot development between control and pGmLe1AtBBM (Table 5).

TABLE 5
Transformation efficiency and phenotype in transgenic
tomato plants at 75 days after Agrobacterium infection.

| | Transformation | | |
|---|---|---|---|
| Construct | Frequency | Shoot Development | Root Growth |
| control | +++ | ++ | − |
| pGmLe1AtBBM | +++ | ++ | − |
| pGmLe1AtWUS | ++++ | ++++ | − |

− No plants with complete root and shoot regenerated as of data recording
+ Fair
++ Moderate
+++ High
++++ Highest

The plant phenotype of M82 plants transformed with pGmLe1AtWUS from earlier experiments was normal.

### Example 5

### Use of Transcription Factor Genes for Transformation in Cassava

### Cassava Transformation

Agrobacterium-mediated transformation was performed using friable embryogenic callus (FEC) of cassava accessions 60444, TME 419 and 91/02324, with subsequent plant regeneration. Somatic embryos were induced from leaf lobes of in vitro micropropagated plants by culture on Murashige and Skoog (MS) basal medium supplemented with 20 g/l sucrose (MS2) plus 50 μM picloram. Pre-cotyledon stage embryos were subcultured onto Gresshoff and Doy basal medium (GD) supplemented with 20 g/l sucrose (MS2) 50 μM picloram (GD2 SOP) to induce production of FECs.

Homogenous FECs were selected and used as target tissue for transformation with A. tumefaciens strain AGL1 carrying each of 5 TF constructs described in example 2. One day before transformation, AGL1 strains were streaked onto solid LB medium containing 200 AM AS and placed in an incubator overnight at 26-28° C. On the day of transformation, Agrobacteria were diluted into 50 mL conical tubes containing liquid GD2 50P medium supplemented with 200 AM AS to attain an OD 600 nm reading up to 0.75. For infection, FECs were placed into 50 mL conical tubes containing Agrobacterium suspension and underwent sonication with a Branson 3510-DTH Ultrasonic Cleaner for 3 seconds. Afterwards the tubes were placed on a shaker for 3 to 4 h at room temperature. The Agrobacterium suspension was then drawn off from the Petri dish and the infected FEC were spread onto 2 layers of Whatman Grade 1 Qualitative Filter Paper (70 mm diameter) on a new Petri dish. The top layer of filter paper containing infected FEC was placed onto a Petri dish containing semi-solid GD2 50P medium supplemented with 200 μm AS. After 3 to 5 days co-cultivation at 21° C., Agrobacterium was then controlled by the addition of 200 mg/l cefotaxime to GD2 50P basal medium and MS2 medium. Transgenic cells were selected and proliferated on GD2 50P containing paromomycin, prior to regeneration of embryos on MS2 medium supplemented with naphthalene acetic acid (NAA). Somatic embryos were germinated on MS2 medium containing BAP and regenerated plants maintained on MS2.

Ten to fourteen days after spreading onto selection medium, actively growing FEC groups larger than 2-3 mm in diameter were identified under the dissection microscope and subcultured using fine forceps onto Stage 1 regeneration medium consisting of MS2 medium supplemented with 5 μM NAA, 200 mg/l cefotaxime and 45 μM paromomycin, with 6 to 10 calli placed onto each Petri dish. This process was repeated 10 and 20 days later, with growing FEC units removed from the GD2 50P selection plates at each time and transferred to Stage 1 regeneration medium. After 21 days culture on Stage 1 medium, actively growing FEC colonies were subcultured onto Stage 2 regeneration medium consisting of MS2 medium supplemented with 0.5 μM NAA and 45 μM paromomycin. After 21 days culture on Stage 2 regeneration medium, green cotyledon-stage embryos were selected from each putative transgenic line and 5 to 6 embryos individually transferred to a Petri dish containing MS2 medium supplemented with 2 μM BAP (MS2 2BAP). Germination of somatic embryos to produce a distinct stem and tri-lobed leaves occurred in an asynchronous manner over the following 3 to 4 weeks. Once distinct shoots were formed, they were excised from the juvenile cotyledon tissues and transferred to MS2 medium to obtain stem elongation and plantlet establishment. After three to 4 weeks culture on MS2 medium, the more robust plant was retained and micropropagated.

### Effects of BABYBOOM (BBM) and WUSCHEL (WUS) on Transformation Frequency and Plant Phenotype in Transgenic Cassava

Cassava plants transformed with pGmLe1AtWUS and pGmLe1AtBBM showed a normal phenotype.

### Example 6

### Use of Transcription Factor Genes for Transformation in Soybean

### Soybean Transformation

Soybean transformation was performed using immature cotyledons and embryogenic suspension cultures of Williams 82 via microparticle bombardment. Immature pods were removed when seeds were 3 to 7 mm long and surface-sterilized for 15 min in 20% Clorox plus 2 drops of Tween 20. After rinsing 3 times in sterile water, immature cotyledons were excised from the pods. After removing the end of the cotyledons containing the embryonic axis, the abaxial surface of each cotyledon half was placed on a solid medium containing MS salts, B5 vitamins, 3% sucrose, 20 mg/l 2,4-D and 0.8% Difco Bacto agar (pH 5.8). Cultures were incubated at 26° C. with 16-hr photoperiods at 50-60 μmol m-² s-¹. After three to six weeks globular somatic embryos were transferred to 30 ml of liquid 10A40N medium supplemented with 15 mM glutamine in a 125 ml Erlenmeyer flask. Embryogenic suspension cultures were maintained on a rotary shaker at 130 rpm and routinely subcultured every month.

A PDS-1000 helium gun (Bio-Rad, Hercules, CA) was used for DNA particle bombardment. About 0.6-0.8 g of embryogenic suspension cultures were placed in the center of a 100 mm diameter Petri dish and the excess liquid was removed. 0.1 to 1 μg of each DNA construct was precipitated, and the precipitates were then resuspended in 85μ of ethanol. For each bombardment, 10 μl of this mixture was loaded on a microcarrier and allowed to dry. A 650-psi rupture pressure disk was used, and sample chamber vacuum was 28 inches of mercury during the bombardment. Bombarded tissues were incubated at 28° C. overnight and were then resuspended in the 10A40N liquid medium. Two weeks after bombardment, the cultures were placed in the same medium but containing 50 mg/l G418. The medium was changed at 2-week intervals. Six to nine weeks after G418 selection, yellow-green outgrowths from the whitish dead clumps were separated and individually transferred for further proliferation into 30 ml of fresh 10A40N medium containing G418. Putative transgenic clones were harvested for YFP expression and were analyzed by polymerase chain reaction (PCR) amplification analysis.

Embryogenic clumps, 2-3 mm in diameter, were transferred to either liquid medium containing MS salts, B5 vitamins, 6% maltose (pH 5.7), or solid medium of the same composition but solidified with 0.2% Phytagel. After two and four weeks of growth in the liquid or solid embryo development medium, respectively, the developing embryos were manually separated and cultured as individual embryos in the fresh solid medium. Individual embryos were kept in Petri dishes for 4 weeks and a portion of the mature embryos were desiccated for 2 to 10 days in empty Petri dishes. Embryos not desiccated were maintained on the same medium. Embryo germination was initiated from the desiccated embryos by transferring to a solid medium containing MS salts, B5 vitamins, 3 sucrose, and 0.2% Phytagel (pH 5.8). After shoot and root development, the plantlets were transferred to Phytatrays containing the same medium for further growth. The regenerated plants with well-developed root systems were then transferred to peat pots (A. H. Hummert Seed Co., St. Louis, MO) containing a sterilized 3:1 soil and vermiculite mixture. Potted plants were placed inside a tray with transparent plastic lid. After complete acclimatization by gradual opening the lid of the tray for 2 weeks, the plants were transferred to the pots and these pots were moved to the greenhouse.

Agrobacterium-mediated transformation was performed using immature cotyledons. A. tumefaciens strain AGL1 carrying each of 5 TF constructs was diluted into 50 ml conical tubes containing liquid infection medium supplemented with 200 μM AS to attain an OD 600 nm reading up to 0.5. For infection, immature cotyledons were placed into 50 ml conical tubes containing Agrobacterium suspension and underwent sonication with a Branson 3510-DTH Ultrasonic Cleaner for 3 seconds. Afterwards the tubes were placed on a shaker for 3 to 4 h at room temperature. The Agrobacterium suspension was then drawn off from the Petri dish and the infected cotyledons were spread onto 2 layers of Whatman Grade 1 Qualitative Filter Paper (70 mm diameter) on a new Petri dish. The top layer of filter paper containing infected cotyledons was placed onto a solid somatic embryo induction medium supplemented with 200 μm AS. After 3 to 5 days co-cultivation at 21° C., Agrobacterium was then controlled by the addition of 200 mg/l cefotaxime to the same medium. After two- to three-week incubation at 26° C., the tissues were moved to 10A40N liquid medium containing 50 mg/l G418. The remaining procedure is the same as the protocol described above for bombardment-mediated transformation.

### Example 7

Use of Baby Boom (BBM) Driven by a Cotyledon-Specific Promoter to Improve Transformation Frequency and Reduce Abnormal Plant Phenotype in Theobroma cacao

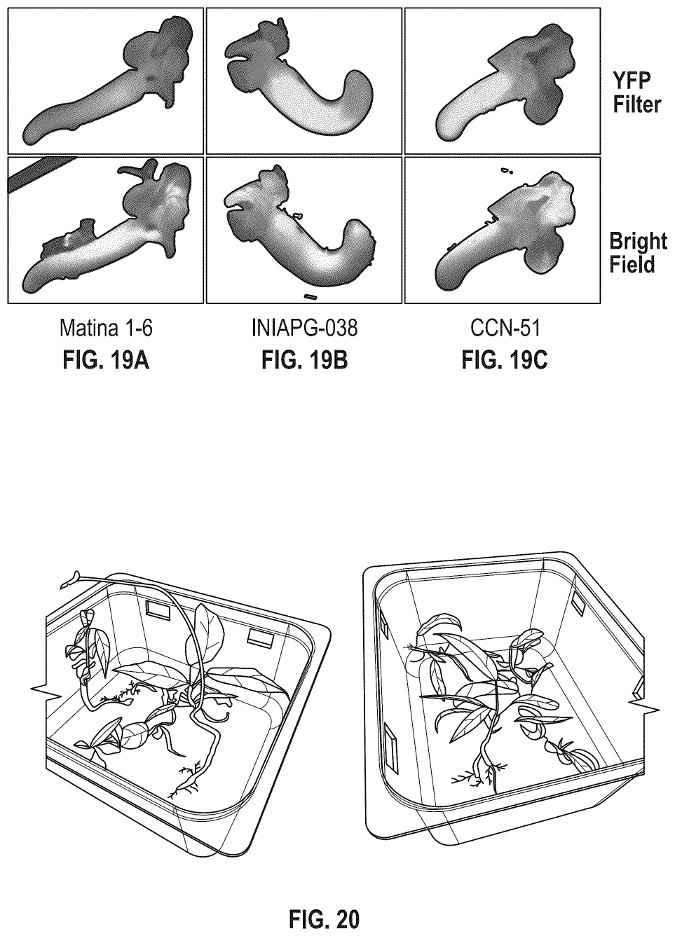

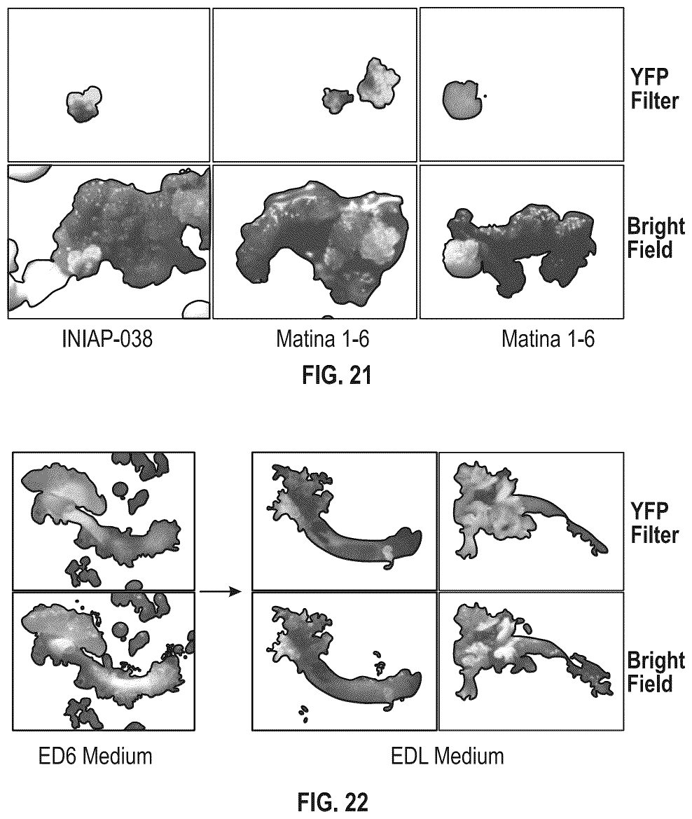

Two sets of transformation experiments were completed to compare the following five constructs on both Matina 1-6 and INIAPG-038: control, pGmLe1AtBBM, pGmLe11AtWUS, pGmLe1VvGRF/GIF and pZmUbi1VvGRF/GIF (Table 7). A single transgenic somatic embryo event in Matina 1-6 was yielded only from pGmLe1AtBBM while no transgenic somatic embryo events were generated from any of 4 other constructs (Table 7). Two transgenic somatic embryo events were generated in INIAPG-038: one from the control construct and another from pGmLe1AtBBM. However, the event from the control construct failed in producing QSEs and eventually plants. In addition, there were no transgenic somatic embryo events generated from any of three other constructs, pGmLe11AtWUS, pGmLe1VvGRF/GIF and pZmUbi1VvGRF/GIF. The single transgenic somatic embryo events from both cultivars transformed with pGmLe1AtBBM were developed into normal mature embryos (FIGS. 19A and 19B), and they then regenerated plants with a normal phenotype (FIG. 20). Stable transformation frequencies using pGmLe1AtBBM at T₀ plant level of Matina 1-6 and INIAPG-038 were 11.9% (1/8.4) and 6.8% (1/14.8), respectively (Table 7). Multiple YFP-expressing events in both Matina 1-6 and INIAPG-038 were generated from pGmLe11AtWUS but all events produced non-regenerable callus tissues (FIG. 21). No regenerable transgenic somatic embryos were also observed from either of GRF/GIF constructs (Table 7).

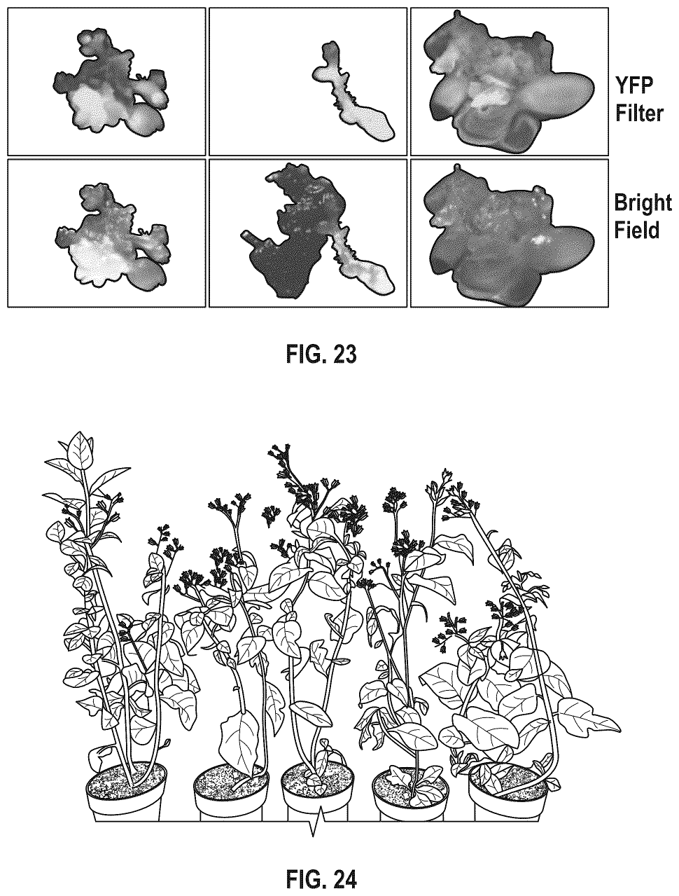

Another set of transformation experiments was conducted on a large scale to compare the control vs pGmLe1AtBBM constructs on Matina 1-6 (Table 7). No transgenic somatic embryo events were generated from cotyledon tissues derived from 680 secondary somatic embryos using the control construct. In contrast, 17 independent YFP-expressing events were generated from pGmLe1AtBBM. These transgenic somatic embryos could be detected 1-2 months after Agrobacterium infection compared to 3-4 months after Agrobacterium infection with the control construct. All except one event could produce quaternary somatic embryos (QSEs) (Table 7). Seven out of 16 QSE-generating events regenerated plants with a normal phenotype, resulting in a T₀ plant level transformation frequency of 6.1% (7 out of 115). The remaining nine QSE-generating events were not regenerable. These events produced multiple somatic embryos on the same tissues with a mildly to moderately abnormal phenotype (FIG. 22 and FIG. 23) and failed germination (FIG. 22) possibly due to too high expression of BBM in the tissues driven by the soybean Le1 (GmLe1) promoter. Similarly, others observed that the introduction of cacao BBM transcription factor driven by the strong constitutive CaMV35S promoter into cacao caused a regeneration issue with an abnormal phenotype even though it significantly increased transformation frequency. They reported that BBM-overexpressing embryos driven by this constitutive promoter never reached normal mature somatic embryo developmental stage or conversion to a new plantlet. Additional transformation experiments were conducted using the pGmLe1AtBBM construct in INIAPG-038 and CCN-51 (Table 7). Two and three regenerable transgenic somatic embryo events (FIG. 19B and FIG. 19C) out of 150 explants of each of INIAPG-038 and CCN-5 were generated, resulting in T₀ plant level transformation frequencies of 1.3% and 2.0%, respectively (Table 7).

In conclusion, a successful Agrobacterium-mediated transformation protocol has been established to produce transgenic Theobroma cacao plants using three elite cultivars, Matina 1-6, INIAPG-038 and CCN-51. The use of moderately strong seed-/cotyledon-/embryo-specific promoter resulted in successful plant regeneration of transgenic BBM-expressing events as well as significantly increased transformation frequency in cacao. Soybean Le1 promoter is known to be a moderately strong seed-specific promoter, but it may be of importance reducing the 10 expression level of BBM to improve the current protocol. Unexpectedly, the overexpression of WUS using the same GmLe1 promoter caused a regeneration issue by producing non-regenerable callus tissues in cacao.

TABLE 7
Stable cacao transformation frequency and plant phenotype using different transcription factor constructs

| | | | # of | | # | | To Normal |
|---|---|---|---|---|---|---|---|
| | | | Somatic | | Events | | Plant |
| | | | Embryo | # | with | To Somatic | Phenotype |
| | | # | Events | Events | Normal | Embryo Level | Level |
| | | Embryos | with YFP | with | Plant | Transformation | Transformation |
| Cultivar | Construct | Infected | Expression | QSE | Phenotype | Frequency | Frequency |
| Matina | Control | 8.4 | 0 | 0 | 0 | 0.0% | 0.0% |
| 1-6 | pGmLe1AtBBM | 8.4 | 1 | 1 | 1 | 11.9% | 11.9% |
| | pGmLe11AtWUS | 8.4 | 0 | 0 | 0 | 0.0% | 0.0% |
| | pGmLe1VvGRF/GIF | 8.4 | 0 | 0 | 0 | 0.0% | 0.0% |
| | pZmUbiVvGRF/GIF | 8.4 | 0 | 0 | 0 | 0.0% | 0.0% |
| INIAPG- | Control | 14.8 | 1 | 0 | 0 | 6.8% | 0.0% |
| 038 | pGmLe1AtBBM | 14.8 | 2 | 1 | 1 | 13.5% | 6.8% |
| | pGmLe11AtWUS | 14.8 | 0 | 0 | 0 | 0.0% | 0.0% |
| | pGmLe1VvGRF/GIF | 14.8 | 0 | 0 | 0 | 0.0% | 0.0% |
| | pZmUbiVvGRF/GIF | 14.8 | 0 | 0 | 0 | 0.0% | 0.0% |
| Matina | Control | 680 | 0 | 0 | 0 | 0.0% | 0.0% |
| 1-6 | | | | | | | |
| Matina | pGmLe1AtBBM | 115 | 17 | 16 | 7 | 14.8% | 6.1% |
| 1-6 | | | | | | | |
| INIAPG- | pGmLe1AtBBM | 150 | 11 | 2 | 2 | 7.3% | 1.3% |
| 038 | | | | | | | |
| CCN 51 | pGmLe1AtBBM | 150 | 6 | 6 | 3 | 4.0% | 2.0% |

### Example 8

### Effects of BABYBOOM (BBM) and WUSCHEL (WUS) on Plant Phenotype in Transgenic Tobacco

Further to the data presented in Example 3 above, no significant difference in plant phenotype among tobacco plants transformed with control, pGmLe1AtBBM or pGmLe1AtWUS (FIG. 24) was observed.

### Example 9

### Effects of BABYBOOM (BBM) and WUSCHEL (WUS) on Transformation Frequency and Plant Phenotype in Transgenic Tomato

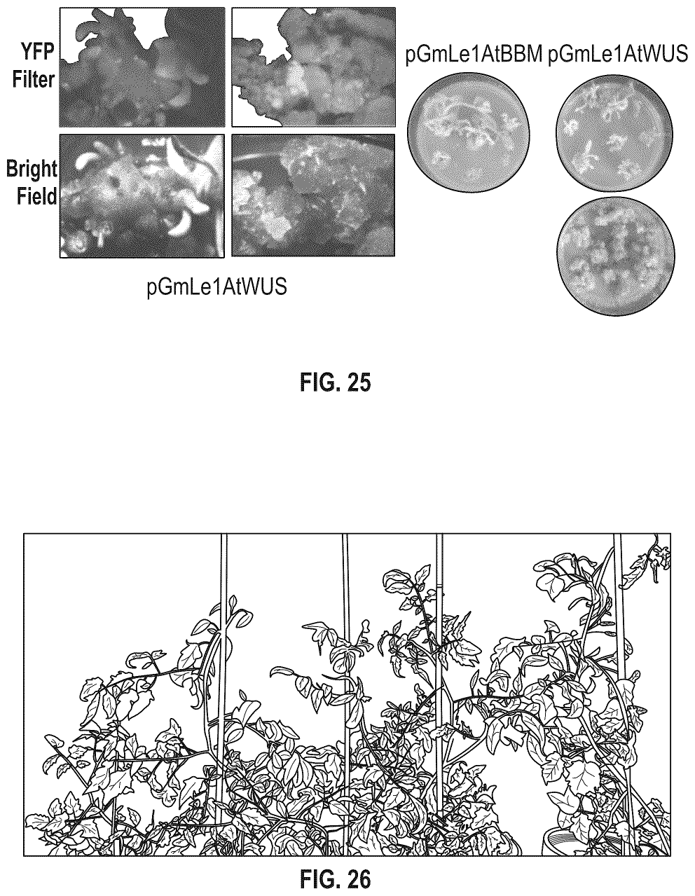

Further to the data presented in Example 4 above, a high percentage of green callus-type tissue was observed from the pGmLe1AtWUS-infected treatment (FIG. 25). They were not regenerable or initially slow in regeneration. However, this type of tissue was not much produced from pGmLe1AtBBM and control constructs. Stable transformation frequencies at T₀ plant level of M82 using pGmLe1AtBBM, pGmLe1AtWUS and control constructs were 43.3% (13/30), 36.7% (11/30) and 13.3% (4/30), respectively (Table 8). pGmLe1AtBBM and pGmLe1AtWUS were 3.3- and 2.8-fold higher than control, respectively. Regenerated M82 tomato plants transformed with pGmLe1AtWUS and pGmLe1AtBBM had a normal phenotype (FIG. 26).

TABLE 8
Stable tomato transformation frequency and plant phenotype
using different transcription factor constructs

| | | # Explants | # Events | |
|---|---|---|---|---|
| | | (Half | with Plant | To Plant Level |
| | | Cotyledons) | Regeneration/ | Transformation |
| Cultivar | Construct | Infected | Rooting | Frequency |
| M82 | control | 30 | 4 | 13.3% |
| M82 | pGmLe1AtBBM | 30 | 13 | 43.3% |
| M82 | pGmLel AtWUS | 30 | 11 | 36.7% |

### Example 10

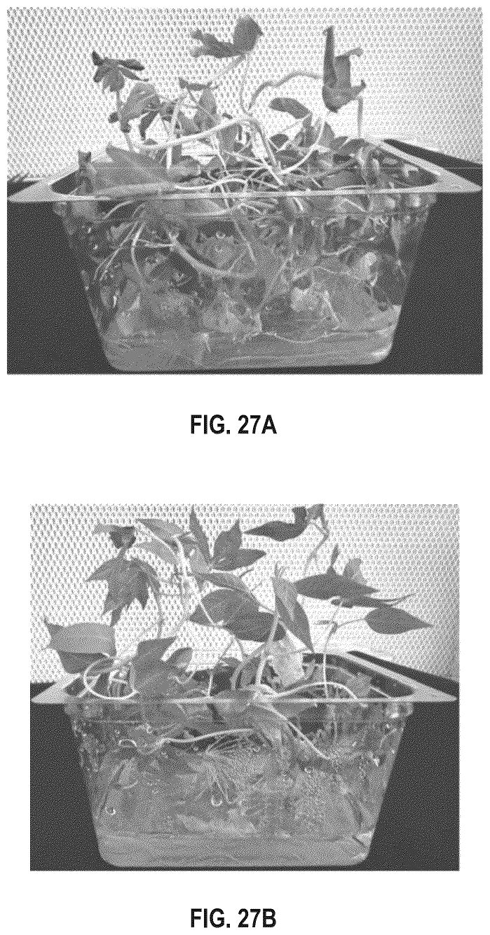

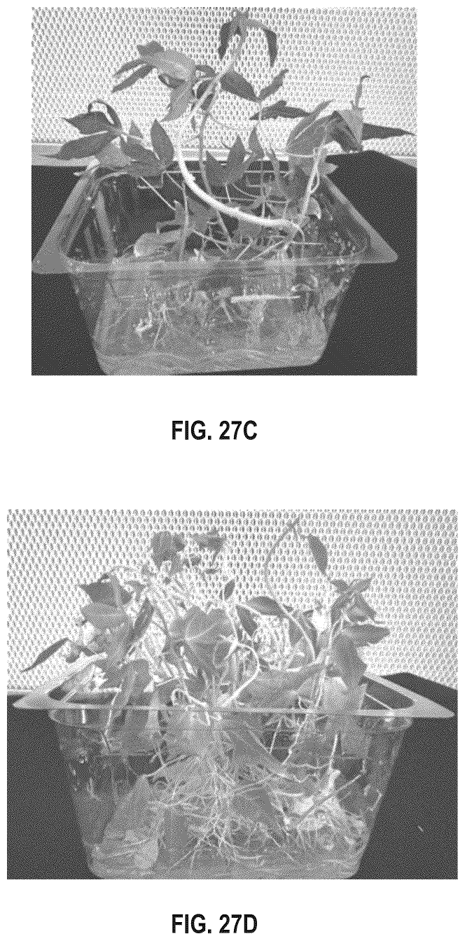

Further to the data presented in Example 5 above, approximately one gram (3-4 pieces of FECs) of homogenous cassava (cv. 60444) FECs was selected and used as target tissue for transformation with A. tumefaciens strain AGL1 carrying each of five different TF constructs described in Example 2. The construct with the highest transformation frequency at T₀ plant level was pGmLe1AtBBM (Table 9). pGmLe11AtBBM produced 14 regenerable events and it was 7-fold higher than the control construct yielding 2 events. Only one single event was produced from either pGmLe1AtWUS or pZmUbi1VvGRF/GIF (Table 9). No regenerable transgenic event was observed from pGmLe1VvGRF/GIF. Regenerated cassava plants transformed pGmLe1AtBBM, pGmLe1AtWUS, and pZmUbi1VvGRF/GIF appeared to have a normal phenotype (FIGS. 27A-27D). However, the phenotype of pGmLe1VvGRF/GIF transformants has not been evaluated because we have not produced fully regenerated plants from these constructs yet (Table 9).

TABLE 9
Stable cacao transformation frequency and plant phenotype
using different transcription factor constructs

| | | | Total # of | Total # of | | |
|---|---|---|---|---|---|---|
| | | | Events | Events | # PCR- | |
| | | | Recovered | Recovered | positive | |
| | | Amount | on Stage 2 | on | Events with | Plant |
| | | of FECs | Selection | Germination | Plant | Pheno- |
| Cultivar | Construct | Infected | Medium | Medium | Regeneration | type |
| 60444 | Control | 1 g | 50 | 10 | 2 | Normal |
| | pGmLe1AtBBM | 1 g | 130 | 45 | 14 | Normal |
| | pGmLe1AtWUS | 1 g | 28 | 20 | 1 | Normal |
| | pGmLe1VvGRF/GIF | 1 g | 15 | 7 | 0 | N/A |
| | pZmUbiVvGRF/GIF | 1 g | 21 | 13 | 1 | Normal |

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-38. (canceled)

39. A transformed dicotyledon or gymnosperm plant cell comprising a heterologous nucleic acid construct comprising a plant seed-specific promoter and a morphogenic gene transcription factor BabyBoom (BBM).

40. The transformed plant cell of claim 39, wherein the plant cell is a dicotyledon.

41. The transformed plant cell of claim 39, wherein the plant cell is a gymnosperm cell.

42. The transformed plant cell of claim 39, wherein the promoter is a plant seed-specific promoter.

43. The transformed plant cell of claim 42, wherein the plant seed-specific promoter is a cotyledon seed-specific promoter.

44. The transformed plant cell of claim 43, wherein the cotyledon seed-specific promoter is a seed storage protein gene promoter.

45. The transformed plant cell of claim 43, wherein the cotyledon seed-specific promoter is an embryo-specific promoter.

46. The transformed plant cell of claim 45, wherein the embryo-specific promoter is a cacao seed-specific promoter, a cacao cotyledon-specific promoter, a cacao seed storage protein gene promoter or a cacao embryo-specific promoter.

47. The transformed plant cell of claim 45, wherein the embryo-specific promoter is a soybean seed-specific promoter, a soybean cotyledon-specific promoter, a soybean seed storage protein gene promoter, a soybean embryo-specific promoter, a soybean lectin promoter, soybean glycinin promoter, or a soybean conglycinin promoter.

48. The transformed plant cell of claim 39, wherein the construct further comprises a vector.

49. The transformed plant cell of claim 48, wherein the vector is a plasmid.

50. The transformed plant cell of claim 39, wherein the construct further comprises a site-specific recombinase.

51. The transgenic plant cell of claim 39, wherein the construct further comprises a trait gene.

52. The transgenic plant cell of claim 39, wherein the construct further comprises a selectable/visual marker gene.

53. The transgenic plant cell of claim 39, wherein the construct further comprises a CRISPR cassette.

54. A nucleic acid construct comprising a plant seed-specific promoter and a morphogenic gene transcription factor BabyBoom (BBM).

55. A method of transforming dicotyledon or gymnosperm plant tissue, comprising contacting the plant tissue with a heterologous nucleic acid construct comprising the nucleic acid construct of claim 54.

56. The method of claim 55, wherein the plant tissue is dicotyledon tissue.

57. The method of claim 55, wherein the tissue is gymnosperm tissue.

58. The method of claim 55, wherein the plant tissue is zygotic embryo, a somatic embryo, somatic embryogenic callus tissue, organogenic tissue, shoot meristematic tissue, a geminating seedling, leaf tissue, or protoplast-derived tissue.

59. A nucleic acid construct comprising pGmLe1AtBBM.
